(19) United States
(12) Patent Application Publication
(10) Pub. No.: US 2021/0259281 A1
(43) Pub. Date: Aug. 26, 2021

(54) Pulse Protein Isolation by Ultrafiltration

(71) Applicant: Eat JUST, Inc., San Francisco, CA (US)

(72) Inventors: Aniket Kale, Pleasanton, CA (US); Meng Li, San Francisco, CA (US); Akihiro Takino, San Francisco, CA (US); Pavan Kambam, Cupertino, CA (US)

(21) Appl. No.: 17/186,931

(22) Filed: Feb. 26, 2021

Related U.S. Application Data
(60) Provisional application No. 62/981,890, filed on Feb. 26, 2020.
(60) Provisional application No. 63/018,692, filed on May 1, 2020.

(51) Int. Cl. A23J 1/14; A23L 33/185; B01D 61/14; B01D 61/16; B01D 69/02; C07K 14/415; C07K 1/36; C07K 1/14; C07K 1/34

(52) U.S. Cl. CPC A23J 1/14; B01D 2315/16; B01D 61/145; B01D 61/16; B01D 69/02; C07K 14/415; C07K 1/36; C07K 1/145; C07K 1/34; A23V 2002/00; B01D 2311/04; B01D 2311/103; B01D 2311/18; B01D 2311/2649; B01D 2325/02; B01D 2325/20; A23L 33/185

(57) ABSTRACT

Pulse protein isolates, food compositions containing such isolates, and methods for preparing pulse protein isolates are disclosed. In some embodiments, the methods include extracting pulse proteins from a milled composition and applying the extracted proteins to an ultrafiltration process to produce pulse protein isolates with desirable organoleptic characteristics.

### CROSS-REFERENCE TO RELATED APPLICATIONS

This application claims the benefit under 35 USC § 119(e) of US Provisional Application Nos. 62/981,890, filed Feb. 26, 2020, and 63/018,692, filed May 1, 2020, each of which is incorporated herein by reference in its entirety for all purposes.

### FIELD OF THE INVENTION

The present disclosure relates to pulse protein isolation, and to pulse protein isolates and uses and compositions thereof.

### BACKGROUND

Use of plant-based proteins such as soy and pea as animal protein substitutes have garnered increasing attention as consumers seek alternatives to conventional animal-based products to reduce the environmental impacts of animal husbandry and to improve dietary options that minimize the negative implications of consuming many animal protein products.

Conventional methods and processes used for extracting plant protein isolates and concentrates include alkaline extraction and acid precipitation (wet process), as well as air classification (dry process). The quality of the plant protein compositions produced by these methods is directly dependent on the operating conditions used to prepare them. Application of an acidic, alkaline or neutral extraction process directly influences functional properties, e.g., the gelling, foaming or emulsifying properties of the protein compositions obtained, which makes the resulting protein compositions unsuitable for certain applications. It may therefore be necessary to modify the protein compositions so as to confer desired properties in the context of food applications. Thus, there remains a need for processes of isolating plant-based proteins with physical characteristics and organoleptic properties desirable for the production of food products, including alternatives to conventional products containing animal proteins.

### BRIEF SUMMARY OF THE INVENTION

In one aspect, the present disclosure provides a method for preparing a pulse protein isolate, comprising: extracting protein from a milled composition (flour) comprising pulse proteins in an aqueous solution at a pH of from about 1 to about 9 to produce a protein rich fraction containing extracted pulse proteins; applying the protein rich fraction to an ultrafiltration process comprising a semi-permeable membrane to separate a retentate fraction from a permeate fraction based on molecular size at a temperature of from 2° C. to 60° C.; and collecting the retentate fraction containing the pulse protein isolate.

In one embodiment, the milled composition is air classified to separate denser flour particles from the less dense particles to prepare air-classified flour, prior to the aqueous extraction step for producing the protein rich fraction containing extracted pulse proteins.

In another embodiment, the protein rich fraction containing the extracted pulse proteins is pre-filtered prior to the ultrafiltration process.

In some embodiments, the method further comprises adjusting the pH of the retentate fraction to a first pH of from 4.0 to 7.0, optionally followed by a further pH adjustment to a second pH of from 5.0 to 6.6. In some cases, the first pH or the second pH of the retentate fraction is adjusted to a pH of from 5.8 to 6.6. In some cases, the first pH or the second pH of the retentate fraction is adjusted to a pH of from 6.0 to 6.2. In some embodiments, the method further comprises heating the retentate fraction to a temperature of from 60° C. to 80° C. for a period of time from 10 seconds to 10 minutes. In some cases, the retentate fraction is heated to a temperature of from 65° C. to 80° C. In some case, the retentate fraction is heated to a temperature of from 70° C. to 80° C. In some case, the retentate fraction is heated to a temperature of from 70° C. to 75° C. In some cases, the retentate fraction is heated for a period of from 10 seconds to 5 minutes. In some cases, the retentate fraction is heated for a period of from 10 seconds to 1 minute. In some cases, the retentate fraction is heated for a period of from 10 seconds to 30 seconds. In some embodiments, the method further comprises removing water from the retentate fraction to produce a concentrated pulse protein isolate. In some cases, removing water from the retentate fraction is performed by spray drying, drum drying, tray drying, ring drying, flash drying or freeze drying. In some embodiments, the method further comprises dehulling pulses, milling pulses, or dehulling and milling pulses to produce the milled composition comprising pulse proteins. In some cases, the pulse is dry-milled. In some cases, the pulse is wet-milled. In some embodiments, the method further comprises drying the pulses prior to milling.

In any embodiments of the methods, the milled composition may comprise dry beans, lentils, faba beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In any embodiments of the methods, the milled composition may comprise Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some cases, the milled composition comprises mung beans (Vigna radiata). In other embodiments, the milled composition may comprise almonds and other nuts, seeds such as sesame seeds, sunflower seeds, and other commonly consumed nuts, fruits and seeds.

In any embodiments, the pulse proteins are not precipitated from the protein rich fraction at a pH of from 4 to 6 or 5 to 6.

In any embodiments of the methods, the retentate fraction comprises pulse proteins having a molecular size of less than 100 kilodaltons (kDa). In some cases, the retentate fraction comprises pulse proteins having a molecular size of less than 50 kDa. In some cases, the retentate fraction comprises pulse proteins having a molecular size of less than 25 kDa. In some cases, the retentate fraction comprises pulse proteins having a molecular size of less than 15 kDa.

In any embodiments of the methods, the permeable membrane may exclude molecules having a size of 10 kDa or larger. In some cases, the permeable membrane excludes molecules having a size of 25 kDa or larger. In some cases, the permeable membrane excludes molecules having a size of 50 kDa or larger. In some cases, the permeable membrane excludes molecules having a size of 1 kDa or larger. In some cases, the permeable membrane excludes molecules having a size of 3 kDa or larger. In some cases, the permeable membrane excludes molecules having a size of 5 kDa or larger. In some cases, the permeable membrane excludes molecules having a size of 7.5 kDa or larger. In some cases, the permeable membrane excludes molecules having a size of 20 kDa or larger. In some cases, the permeable membrane excludes molecules having a size of 30 kDa or larger. In some cases, the permeable membrane excludes molecules having a size of 70 kDa, 80 kDa, 90 kDa, 95 kDa, or larger. In various embodiments, the semi-permeable membrane has a pore size of from 0.001 to 0.1 micron. In some cases, the semi-permeable membrane has a pore size of from 0.001 to 0.006 micron. In some cases, the semi-permeable membrane has a pore size of from 0.001 to 0.005 micron. In some cases, the semi-permeable membrane has a pore size of from 0.0025 to 0.005 micron. In some cases, the semi-permeable membrane has a pore size of about 0.003 micron.

In any embodiments of the methods, the permeable membrane may be a polymeric membrane, a ceramic membrane, or a metallic membrane. In various embodiments, the semi-permeable membrane is made from polyvinylidine fluoride (PVDF), polyether sulfone (PES), polyacrylonitrile (PAN), polytetrafluoroethylene (PTFE), polyamide-imide (PAI), a natural polymer, rubber, wool, cellulose, stainless steel, tungsten, palladium, an oxide, a nitride, a metallic carbide, aluminum carbide, titanium carbide, or a hydrated aluminosilicate mineral containing an alkali and alkaline-earth metal.

In any embodiments of the methods, the ultrafiltration process is performed at a pressure of from about 20 to about 500 psig.

In any embodiments of the methods, the aqueous solution may comprise a salt. In some embodiments, the aqueous solution may comprise a salt at a concentration of at least 0.1% w/v. In some cases, the aqueous solution comprises a salt at a concentration of from 0.01% w/v to 5% w/v.

In some cases, the aqueous solution comprises a salt at a concentration of from 0.001% w/v to 0.1% w/v, 0.001% w/v to 0.2% w/v, 0.001% w/v to 0.3% w/v, or 0.001% w/v to 0.4% w/v. In some cases, the aqueous solution comprises a salt at a concentration of from 0.1% w/v to 0.5% w/v, 0.1% w/v to 1.0% w/v, 1.0% w/v to 2.5% w/v, or 2.5% w/v to 5% w/v. In various embodiments, the salt is selected from sodium chloride, sodium sulfate, sodium phosphate, ammonium sulfate, ammonium phosphate, ammonium chloride, potassium chloride, potassium sulfate, or potassium phosphate. In some cases, the salt is NaCl. In some embodiments, the aqueous solution does not comprise a salt.

In any of the various embodiments of the methods, the density of the pulse protein isolate is less than 0.6 g/ml. In some cases, the density of the pulse protein isolate is less than 0.5 g/ml or less than 0.4 g/ml. In any of the various embodiments of the methods, the density of the pulse protein isolate is less than 0.3 g/ml. In some cases, the density of the pulse protein isolate is less than 0.2 g/ml or less than 0.1 g/ml.

In any of the various embodiments of the methods, a homogenized protein dispersion consisting of 12% w/w of the pulse protein isolate, 0.35% w/w NaCl, and water has a separation ratio of less than 30% after 48 hours of storage at 4° C. In some cases, the separation ratio is less than 25% after 48 hours of storage at 4° C. In some cases, the separation ratio is less than 20% after 48 hours of storage at 4° C.

In any of the various embodiments of the methods, the pulse protein isolate has a storage modulus of from 25 Pa to 500 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s. In any of the various embodiments of the methods, the pulse protein isolate has a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s.

In any of the various embodiments of the methods, the pulse protein isolate has a linear viscoelastic region of from 25 Pa to 1500 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In any of the various embodiments of the methods, the pulse protein isolate has a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In some cases, the pulse protein isolate has a linear viscoelastic region of less than 500 Pa at up to 10% strain, or a linear viscoelastic region of less than 200 Pa at up to 10% strain.

In another aspect, the present disclosure provides a pulse protein isolate prepared by any one of the methods discussed above or herein.

In another aspect, the present disclosure provides a food composition comprising a pulse protein isolate discussed above or herein, and one or more edible ingredients.

In another aspect, the present disclosure provides an isolated pulse protein having a density of less than 0.6 g/ml. In some cases, the isolated pulse protein has a density of less than 0.5 g/ml or less than 0.4 g/ml. In some embodiments, the isolated pulse protein has a density of less than 0.3 g/ml. In some cases, the isolated pulse protein has a density of less than 0.2 g/ml or less than 0.1 g/ml.

In another aspect, the present disclosure provides an isolated pulse protein having a storage modulus of from 25 Pa to 500 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s. In another aspect, the present disclosure provides an isolated pulse protein having a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s.

In another aspect, the present disclosure provides an isolated pulse protein having a linear viscoelastic region of from 25 Pa to 1500 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In another aspect, the present disclosure provides an isolated pulse protein having a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In some cases, the pulse protein has a linear viscoelastic region of less than 500 Pa at up to 10% strain, or a linear viscoelastic region of less than 200 Pa at up to 10% strain.

In any of the various embodiments of the isolated pulse protein, the pulse protein may have been isolated from dry beans, lentils, faba beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In any of the various embodiments of the isolated pulse protein, the pulse protein may be isolated from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some cases, the pulse protein is isolated from mung beans (Vigna radiata). In other embodiments, the milled composition may comprise almonds and other nuts, seeds such as sesame seeds, sunflower seeds, and other commonly consumed nuts, fruits and seeds.

In any of the various embodiments of the isolated pulse protein, the pulse protein may include proteins having a molecular size of less than 100 kDa. In some embodiments, the pulse protein includes proteins having a molecular size of less than 50 kDa. In some embodiments, the pulse protein includes proteins having a molecular size of less than 25 kDa. In some embodiments, the pulse protein includes proteins having a molecular size of less than 15 kDa. In some embodiments, the pulse protein includes proteins having a molecular size of from 1 kDa to 99 kDa.

In another aspect, the present disclosure provides a food composition comprising a pulse protein isolate as discussed above or herein, and one or more edible ingredients. In some embodiments, the composition has a viscosity of less than 500 cP after storage for thirty days at 4° C. In some embodiments, the composition has a viscosity of less than 500 cP after storage for sixty days at 4° C. In some embodiments, the composition has a viscosity of less than 450 cP after storage for thirty days at 4° C. In some embodiments, the composition has a viscosity of less than 450 cP after storage for sixty days at 4° C.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all intermediate values are encompassed within the scope of the present disclosure.

Other embodiments will become apparent from a review of the ensuing detailed description.

### BRIEF DESCRIPTION OF THE DRAWINGS

FIG. 1 illustrates a process flow for preparation of a pulse protein isolate in accordance with an embodiment of the present invention. The dashed boxes and arrows represent optional process steps.

FIG. 2 illustrates the effects of solid:liquid ratio on protein recovery in the extraction portion of the isolation processes discussed herein. A ratio of about 1:6 yielded near maximum protein recovery while minimizing the volume of liquid for downstream processing.

FIG. 3 illustrates the effects of mean particle size on protein recovery in the extraction portion of the isolation processes discussed herein. A particle size of from 50-200 μm yielded nearly equivalent protein recovery.

FIG. 4 illustrates the effects of pH on protein recovery in the extraction portion of the isolation processes discussed herein. Protein recovery was highest in the pH range 7-9, with greater recovery shown at pH 8.

FIG. 5 illustrates the effects of salt concentration on protein recovery in the extraction portion of the isolation processes discussed herein. No significant variation in protein recovery was observed at salt concentrations varying from 0.1% to 5% w/v at pH 7.0.

FIG. 6 illustrates the combined effects of salt concentration and pH on protein recovery in the extraction portion of the isolation processes discussed herein. Increased concentrations of salt improved protein recovery at acidic pH.

FIGS. 7A and 7B illustrate the densities of pulse protein isolates prepared by isoelectric precipitation and ultrafiltration (FIG. 7A), and the particle size distribution of the same isolates.

FIG. 8 illustrates the separation ratio of protein dispersions made with pulse protein isolates prepared by isoelectric precipitation (IEP19) and ultrafiltration (UF327).

FIGS. 9A and 9B illustrate rheological characterization of pulse protein isolates prepared by isoelectric precipitation (IEP) and ultrafiltration (UF). FIG. 9A shows the storage modulus as a function of temperature for a pulse protein isolate dispersion (12% w/w protein) using isoelectric-precipitated or ultrafiltered isolates, and FIG. 9B shows the storage modulus as a function of oscillation strain for the same dispersions.

FIG. 10 illustrates the viscosity of a food composition (an egg-free liquid egg analog) formulated with pulse protein isolates prepared by isoelectric precipitation (IEP), and (b) ultrafiltration (UF) over a period of sixty days (n=15 for each isolate).

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

As used herein, the singular forms “a,” “an,” and “the” include the plural referents unless the context clearly indicates otherwise.

The term “reduce” indicates a lessening or decrease of an indicated value relative to a reference value. In some embodiments, the term “reduce” (including “reduction”) refers to a lessening or a decrease of an indicated value by about 5%, 10%, 15%, 20%, 25%, 30%, 35%, 40%, 45%, or 50% relative to a reference value. In some embodiments, the term “reduce” (including “reduction”) refers to a lessening or a decrease of an indicated value by at least about 5%, 10%, 15%, 20%, 25%, 30%, 35%, 40%, 45%, or 50% relative to a reference value.

As used herein, the term “eggs” includes but is not limited to chicken eggs, other bird eggs (such as quail eggs, duck eggs, ostrich eggs, turkey eggs, bantam eggs, goose eggs), and fish eggs such as fish roe. Typical food application comparison is made with respect to chicken eggs.

As used herein, the term “enriched,” “increased” or the like refers to an increase in a percent amount of a molecule, for example, a protein, in one sample relative to the amount of the molecule in a reference sample. The enrichment may be conveniently expressed as a percent enrichment or increase. For example, an isolate enriched in a certain type of globulin protein relative to whole pulses (e.g., mung beans) means that, the amount of the globulin protein in the isolate expressed as a percentage of the amount of total protein in the isolate, is higher than the amount of the globulin protein in a whole pulse (e.g., mung bean) expressed as a percentage of the amount of total protein in the whole pulse. In some embodiments, the enrichment is on a weight to weight basis. In some embodiments, the enrichment refers to an increase of about 5%, 10%, 15%, 20%, 25%, 30%, 35%, 40%, 45%, or 50% relative to the reference value or amount. In some embodiments, the enrichment refers to an increase of at least about 5%, 10%, 15%, 20%, 25%, 30%, 35%, 40%, 45%, or 50% relative to the reference value or amount.

As used herein, the term “depleted,” “decreased” or the like refers to a decrease in a percent amount of a molecule, for example, a protein, in one sample relative to the amount of the molecule in a reference sample. The depletion may be conveniently expressed as a percent depletion, decrease or reduction. For example, an isolate decreased in a certain type of globulin protein relative to whole pulses (e.g., mung beans) means that, the amount of the globulin protein in the isolate expressed as a percentage of the amount of total protein in the isolate, is lower than the amount of the globulin protein in a whole pulse (e.g., mung bean) expressed as a percentage of the amount of total protein in the whole pulse. In some embodiments, the depletion is on a weight to weight basis. In some embodiments, the depletion refers to a decrease of about 5%, 10%, 15%, 20%, 25%, 30%, 35%, 40%, 45%, or 50% relative to the reference value or amount. In some embodiments, the depletion refers to a decrease of at least about 5%, 10%, 15%, 20%, 25%, 30%, 35%, 40%, 45%, or 50% relative to the reference value or amount.

As used herein, “molecular weight,” “molecular size” or similar expressions refer to the molecular mass of compounds, such as proteins, expressed as dalton (Da) or kilodalton (kDa). The molecular weight of a compound can be precise or can be an average molecular mass. For example, the molecular weight of a discrete compound, such as NaCl or a specific protein can be precise. For the molecular sizes of protein isolates of the invention, an average molecular mass is typically used. For example, protein isolates obtained in the retentate fraction of a purification process using an ultrafiltration membrane having a molecular weight cut-off of 10 kDa are depleted in proteins (and other compounds) that have an average molecular weight of less 10 kDa. The retentate fraction from a 10 kDa UF membrane can also be described as being enriched in proteins (and other compounds) that have an average molecular weight of greater than 10 kDa. The permeate fraction of a purification process using an ultrafiltration membrane having a molecular weight cut-off of 10 kDa is enriched in proteins (and other compounds) that have an average molecular weight of less than 10 kDa. The permeate fraction from a 10 kDa UF membrane can also be described as being depleted in proteins (and other compounds) that have an average molecular weight of greater than 10 kDa.

As used herein, “plant source of the isolate” refers to a whole plant material such as whole mung bean or other pulse, or from an intermediate material made from the plant, for example, a dehulled bean, a flour, a powder, a meal, ground grains, a cake (such as, for example, a defatted or de-oiled cake), or any other intermediate material suitable to the processing techniques disclosed herein to produce a purified protein isolate.

The term “transglutaminase” refers to an enzyme (R-glutamyl-peptide:amine glutamyl transferase) that catalyzes the acyl-transfer between γ-carboxyamide groups and various primary amines, classified as EC 2.3.2.13. It is used in the food industry to improve texture of some food products such as dairy, meat and cereal products. It can be isolated from a bacterial source, a fungus, a mold, a fish, a mammal and a plant.

The terms “majority” or “predominantly” with respect to a specified component, e.g., protein content, refer to the component having at least 50% by weight of the referenced batch, process stream, food formulation or composition.

Unless indicated otherwise, percentage (%) of ingredients refer to total % by weight typically on a dry weight basis unless otherwise indicated.

The term “purified protein isolate”, “protein isolate”, “isolate”, “protein extract”, “isolated protein” or “isolated polypeptide” refers to a protein fraction, a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). One or more proteins or fractions may be partially removed or separated from residual source materials and/or non-solid protein materials and, therefore, are non-naturally occurring and are not normally found in nature. A polypeptide or protein may also be rendered substantially free of naturally associated components by isolation, using protein purification techniques known in the art and as described herein. A polypeptide that is chemically synthesized or synthesized in a cellular system different from the cell from which it naturally originates will be “isolated” from its naturally associated components. As thus defined, “isolated” does not necessarily require that the protein, polypeptide, peptide or oligopeptide so described has been physically removed from its native environment.

### Methods of Producing Pulse Protein Isolates

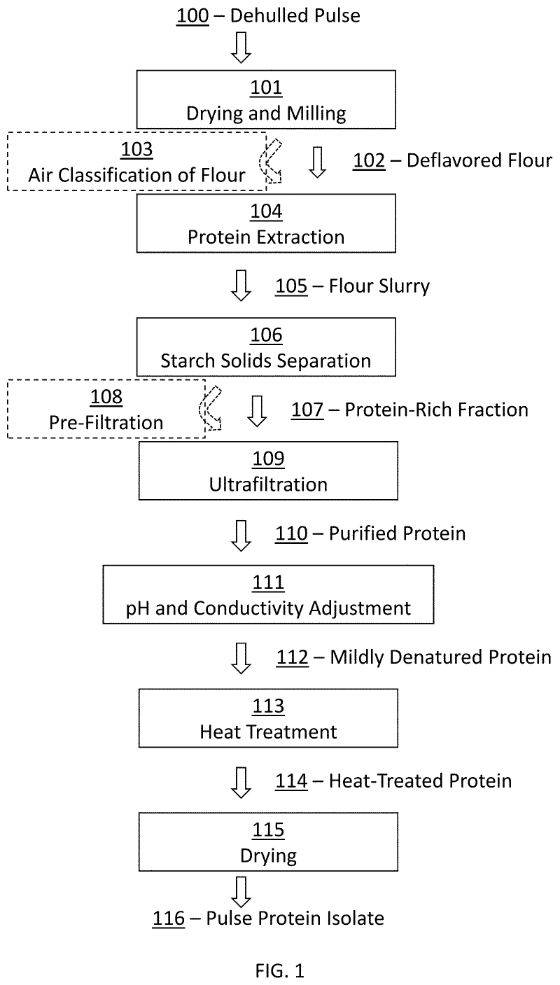

The present disclosure includes methods of preparing pulse protein isolates (e.g., mung bean protein isolates) using ultrafiltration techniques. The pulse protein isolates prepared by these methods have characteristics that are advantageous for the preparation of food product compositions, as discussed in greater detail below. An exemplary embodiment of a method for producing pulse protein isolates is shown in FIG. 1, and includes drying and milling (101) a dehulled pulse (100) to produce a deflavored flour (102), which is then subjected to protein extraction (104) to produce a flour slurry (105). Starch solids are separated (106) from the flour slurry to produce a protein-rich fraction (107) that is then introduced into an ultrafiltration process (109) to produce a purified protein (110).

In one embodiment, the deflavored flour (102) is air classified (103) prior to protein extraction (104) to produce the flour slurry (105). Air classification separates denser flour particles from the less dense particles. Less dense flour particles are higher in protein content than higher density particles.

In one embodiment, the protein-rich fraction (107) is pre-filtered (108) to remove residual solids remaining in the protein-rich fraction prior to the ultrafiltration step. Pre-filtration (108) may extend the usable lifetime of the ultrafiltration membrane by reducing clogging of the ultrafiltration membrane. Pre-filtration can be accomplished by use of a pressure based filtration method such as microfiltration or use of filters that can exclude very large molecular compounds, e.g. molecules of greater than 500 kDa. When using microfiltration, a micro-filter having a pore size of between 0.1-100 microns prior to the ultrafiltration process (109) can be utilized. Similarly, vacuum based pre-filtration such as rotary vacuum-drum filtration can used. Alternatively, centrifugal pre-filtration such as decanter centrifuges, disc stack centrifuges can be used. The purified protein product (110) is then adjusted for pH and conductivity (111) to produce a mildly denatured protein (112) that is then subjected to heat treatment (113) for pasteurization, and the heat-treated protein (114) is dried (115) to produce the pulse protein isolate (116).

In some embodiments, the methods for producing the pulse protein isolate comprise (a) extracting protein from a milled composition comprising pulse proteins in an aqueous solution at a pH of from about 1 to about 9 to produce a protein rich fraction containing extracted pulse proteins, (b) applying the protein rich fraction to an ultrafiltration process comprising a semi-permeable membrane to separate a retentate fraction from a permeate fraction based on molecular size at a temperature of from about 5° C. to about 60° C., (c) collecting the retentate fraction containing the pulse protein isolate. In various embodiments, the methods may further comprise: dehulling and milling pulses to produce the milled composition comprising pulse proteins; drying the pulses prior to milling; adjusting the pH and/or conductivity of the retentate fraction; heating the retentate fraction to pasteurize the pulse proteins; and/or removing water or drying the retentate fraction and/or the pulse protein isolate.

In various embodiments, the pulse proteins may be isolated from any pulse, including dry beans, lentils, faba beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In various embodiments, the pulse proteins may be isolated from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse proteins are isolated from mung beans (Vigna radiata). In other embodiments, the milled composition may comprise almonds and other nuts, seeds such as sesame seeds, sunflower seeds, and other commonly consumed nuts, fruits and seeds.

In various embodiments, the methods discussed above or herein produce a pulse protein isolate comprising pulse proteins having a molecular size of less than 100 kilodaltons (kDa). In some embodiments, the methods produce a pulse protein isolate comprising pulse proteins having a molecular size of less than 95 kDa, 90 kDa, 85 kDa, 80 kDa, 75 kDa, 70 kDa, 65 kDa, 60, kDa, 55 kDa, 50 kDa, 45 kDa, 40 kDa, 35 kDa, 30 kDa, 25 kDa, 20 kDa or 15 kDa. In various embodiments, the methods produce a pulse protein isolate comprising pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the methods discussed above or herein produce a pulse protein isolate comprising pulse proteins enriched in proteins having a molecular size of greater than 5 kilodaltons (kDa). In some embodiments, the methods produce a pulse protein isolate comprising pulse proteins enriched in proteins having a molecular size of greater than 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa or 95 kDa. In some embodiments, the methods produce a pulse protein isolate comprising pulse proteins enriched in proteins having a molecular size of less than 100 kDa.

In some embodiments, the methods produce a pulse protein isolate comprising pulse proteins enriched in proteins having a molecular size of from 1 kDa to 99 kDa, from 1 kDa to 75 kDa, from 1 kDa to 50 kDa, from 1 kDa to 25 kDa, from 5 kDa to 99 kDa, from 5 kDa to 75 kDa, from 5 kDa to 50 kDa, from 5 kDa to 25 kDa, from 10 kDa to 99 kDa, from 10 kDa to 75 kDa, from 10 kDa to 50 kDa, or from 10 kDa to 25 kDa. In various embodiments, the methods produce a pulse protein isolate comprising pulse proteins, or enriched in pulse proteins, having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the methods discussed above or herein produce a pulse protein isolate comprising pulse proteins depleted in proteins having a molecular size of less than 5 kilodaltons (kDa). In some embodiments, the methods produce a pulse protein isolate comprising pulse proteins depleted in proteins having a molecular size of less than 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa or 95 kDa. In various embodiments, the methods produce a pulse protein isolate comprising pulse proteins, or enriched in pulse proteins, having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the methods discussed above or herein produce a pulse protein isolate comprising pulse proteins having a density of less than 0.3 g/ml.

In various embodiments, the methods discussed above or herein produce a pulse protein isolate comprising pulse proteins that, when formulated into a homogenized protein dispersion consisting of 12% w/w pulse protein, 0.35% w/w NaCl, and water has a separation ratio of less than 30% after 48 hours of storage at 4° C. In some embodiments, the methods produce a pulse protein isolate comprising pulse proteins that, when formulated into a homogenized protein dispersion consisting of 12% w/w pulse protein, 0.35% w/w NaCl, and water has a separation ratio of less than 25% after 48 hours of storage at 4° C. In some embodiments, the methods produce a pulse protein isolate comprising pulse proteins that, when formulated into a homogenized protein dispersion consisting of 12% w/w pulse protein, 0.35% w/w NaCl, and water has a separation ratio of less than 20% after 48 hours of storage at 4° C.

In various embodiments, the methods discussed above or herein produce a pulse protein isolate having a storage modulus of from 25 Pa to 500 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s. In various embodiments, the methods discussed above or herein produce a pulse protein isolate having a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s.

In various embodiments, the methods discussed above or herein produce a pulse protein isolate having a linear viscoelastic region of from 25 Pa to 1500 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In various embodiments, the methods discussed above or herein produce a pulse protein isolate having a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In some embodiments, the methods produce a pulse protein isolate having a linear viscoelastic region of less than 500 Pa at up to 10% strain, or a linear viscoelastic region of less than 200 Pa at up to 10% strain.

### Dehulling, Drying and Milling

The pulse protein isolates (e.g., mung bean isolates) provided herein may be prepared from any suitable source of pulse protein, where the starting material is whole plant material (e.g., whole mung bean). In some cases, the methods may include dehulling the raw source material. In some such embodiments, raw pulse protein materials (e.g., mung beans) may be de-hulled in one or more steps of pitting, soaking, and drying to remove the seed coat (husk) and pericarp (bran). The de-hulled material (e.g., mung beans) are then milled to produce a composition (e.g., flour) with a well-defined particle distribution size. The types of mills employed may include one or a combination of a hammer, pin, knife, burr, and air classifying mills.

Air classification is an industrial process in which materials are separated by a combination of density, size and/or shape. Dried materials such as pulse flours, for example mung bean flour, are introduced into an air classifier (air elutriator) where the flour particles are subjected to a column of rising air. The less dense flour particles are carried further in the air stream and separation of flour particles by density is achieved. The applicant has discovered that less dense pulse flour particles contain higher amounts of protein than the flour particles with higher density.

### Protein Extraction

The methods for producing the pulse protein isolate comprise extracting protein from a milled composition comprising pulse proteins in an aqueous solution at a pH of from about 1 to about 9 to produce a protein rich fraction containing extracted pulse proteins. In some embodiments, the aqueous solution has a pH of from about 4 to about 9. In some embodiments, the aqueous solution has a pH of from about 6 to about 10. In some embodiments, the aqueous solution has a pH of about 7 to about 9. In some embodiment, the aqueous solution has a pH of about 8. In various embodiments, the pH of the aqueous solution is about 1, 1.5, 2, 2.5, 3, 3.5, 4, 4.5, 5, 5.5, 6, 6.5, 7, 7.5, 8, 8.5, 9, 9.5 or 10. In some embodiments, the extraction is performed at a pH of 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5. The pH of the slurry may be adjusted with, e.g., a food-grade 50% sodium hydroxide solution to reach the desired extraction pH.

In some embodiments of the extraction step, an intermediate starting material, for example, a milled composition comprising pulse proteins (e.g., mung bean flour), is mixed with an aqueous solution to form a slurry. In some embodiments, the aqueous solution is water, for example soft water. The aqueous extraction may include creating an aqueous solution comprising one part of the source of the plant protein (e.g., flour) to about, for example, 3 to 15 parts aqueous extraction solution. Additional useful solid:liquid ratios for extraction include 1:2, 1:3, 1:4, 1:5, 1:6, 1:7, 1:8, 1:9, 1:10, 1:11, 1:12, 1:13, 1:14, 1:15. In some embodiments, extraction is performed using a solid:liquid ratio of 1:6.

In some cases, the aqueous solution comprises a salt. In some cases, the salt concentration is at least 0.01% w/v. In some cases, the salt concentration is at least 0.1% w/v. In some cases, the salt concentration is from 0.01% w/v to 5% w/v. In various embodiments, the salt concentration is 0.001%, 0.0025%, 0.005%, 0.0075%, 0.01%, 0.025%, 0.05%, 0.075%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0%. In various embodiments, the salt is selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl. In some embodiments, the aqueous solution does not comprise a salt.

In some cases, the aqueous extraction is performed at a desired temperature, for example, about 2-10° C. in a chilled mix tank to form the slurry. In some embodiments, the mixing is performed under moderate to high shear. In some embodiments, a food-grade de-foaming agent (e.g., KFO 402 Polyglycol) is added to the slurry to reduce foaming during the mixing process. De-foamers include, but are not limited to, polyglycol based de-foamers, vegetable oil based de-foamers, and silicone. In other embodiments, a de-foaming agent is not utilized during extraction.

Following extraction, the protein rich fraction may be separated from the slurry, for example, in a solid/liquid separation unit, consisting of a decanter and a disc-stack centrifuge. The protein rich fraction may be centrifuged at a low temperature, e.g., between 3-10° C. In some cases, the protein rich fraction is collected and the pellet is resuspended in, e.g., 3:1 water-to-protein. The process may be repeated, and the combined protein rich fractions filtered through a Nylon mesh.

### Starch Solids Separation

In some embodiments, the methods may optionally include reducing or removing a fraction comprising carbohydrates (e.g., starches) or a carbohydrate-rich protein isolate, post extraction.

### Charcoal Treatment

Optionally, the protein rich fraction, retentate fraction, or pulse protein isolate may be subjected to a carbon adsorption step to remove non-protein, off-flavor components, and additional fibrous solids from the protein extraction. This carbon adsorption step leads to a clarified protein extract. In one embodiment of a carbon adsorption step, the protein extract is then sent through a food-grade granular charcoal-filled annular basket column (<5% w/w charcoal-to-protein extract ratio) at 4 to 8° C.

### Ultrafiltration

The methods of the present disclosure utilize ultrafiltration to separate the pulse proteins from other materials. The ultrafiltration process utilizes at least one semi-permeable selective membrane that separates a retentate fraction (containing materials that do not pass through the membrane) from a permeate fraction (containing materials that do pass through the membrane). The semi-permeable membrane separates materials (e.g., proteins and other components) based on molecular size. For example, the semi-permeable membrane used in the ultrafiltration processes of the present methods may exclude molecules (i.e., these molecules are retained in the retentate fraction) having a molecular size of 10 kDa or larger. In some embodiments, the semi-permeable membrane may exclude molecules (e.g., pulse proteins) having a molecular size of 25 kDa or larger. In some embodiments, the semi-permeable membrane excludes molecules having a molecular size of 50 kDa or larger. In various embodiments, the semi-permeable membrane used in the ultrafiltration process of the methods discussed herein excludes molecules (e.g., pulse proteins) having a molecular size greater than 5 kDa, 10 kDa, 15 kDa, 20 kDa, 25 kDa, 30 kDa, 35 kDa, 40, kDa, 45 kDa, 50 kDa, 55 kDa, 60 kDa, 65 kDa, 70 kDa, 75 kDa, 80 kDa, 85 kDa, 90 kDa, or 95 kDa. For example, a 10 kDa membrane allows molecules, including pulse proteins, smaller than 10 kDa in size to pass through the membrane into the permeate fraction, while molecules, including pulse proteins, equal to or larger than 10 kDa are retained in the retentate fraction. An exemplary protocol for the ultrafiltration process is provided in Example 1.

Ultrafiltration (UF) is a cross-flow separation process for separating compounds with particular molecular weights that are present in a liquid. By applying pressure, typically in the range of 20-500 psig to a membrane, the compounds having the specified molecular weight are separated from the liquid. UF membranes have molecular weight cut-off ranges of 1,000 to 500,000 Da. The pore sizes of the membranes typically range between 0.1 to 0.001 micron. The nominal pore size of a UF membrane with a 100 kD cut-off is typically about 0.006 micron and a membrane with a 10 kD cut-off is typically about 0.003 micron. If a liquid solution containing proteins, e.g., mung bean proteins, is subjected to ultrafiltration using a 10 kD membrane, the concentration of proteins having a molecular weight of less than 10 kD is increased in the filtrate (permeate) and decreased in the retentate. Concomitantly, the concentration of proteins having a molecular weight of greater than 10 kD is increased in the retentate and decreased in the filtrate (permeate). In various embodiments of the methods discussed herein, the semipermeable membrane may have a pore size of 0.001, 0.0015, 0.002, 0.0025, 0.003, 0.0035, 0.004, 0.0045, 0.005, 0.0055, or 0.006 micron.

There are various types of UF membranes that are available commercially, including polymeric, ceramic, and metallic membranes having a desired molecular weight cutoff. For polymeric membrane types, these include membranes made from polyvinylidine fluoride (PVDF), polyether sulfone (PES), polyacrylonitrile (PAN), polytetrafluoroethylene (PTFE), polyamide-imide (PAI) and natural polymers including membranes made from rubber, wool, and cellulose. Metallic membranes are made by sintering metal powders onto a porous substrate. Commonly used metal powders are stainless steel, tungsten and palladium. Ceramic membranes are made of oxides, nitrides or carbides of metallic (e.g., aluminum and titanium) and non-metallic materials. UF membranes comprising zeolites are made of hydrated aluminosilicate minerals that contain alkali and alkaline-earth metals. Zeolite UF membranes are useful because of their highly uniform pore size.

The ultrafiltration process of the present methods may be performed at a temperature in a range of from about 5° C. to about 60° C. In some cases, the temperature may be about 15° C., about 20° C., about 25° C., about 30° C., about 35° C., about 40° C., about 45° C., or about 50° C. In some embodiments, the ultrafiltration process is performed at a pressure of from about 20 to about 500 psig. In various embodiments, the ultrafiltration process is performed at a pressure of about 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 psig.

### pH and Conductivity Adjustment

In some embodiments, the methods include adjusting the pH and/or conductivity of the retentate fraction or the pulse protein isolate. In some cases, the pH is adjusted to a range of from about 5.8 to about 6.6. In some embodiment, the pH is adjusted to from 6.0 to 6.2. In various embodiments, the pH is adjusted to 5.8, 5.9, 6.0, 6.1, 6.2, 6.3, 6.4, 6.5 or 6.6. In some embodiments, the conductivity of the retentate fraction or the pulse protein isolate is adjusted. In some embodiments, the conductivity of the retentate fraction or the pulse protein isolate is adjusted to between 1-3 mS/cm using salt if required. In various embodiments, the conductivity is adjusted to 1, 1.1, 1.2, 1.3, 1.4, 1.5, 1.6, 1.7, 1.8, 1.9, 2.0 2.1, 2.2, 2.3, 2.4, 2.5, 2.6, 2.7, 2.8, 2.9, or 3.0 mS/cm. In various embodiments, the salt used to modify the conductivity can be selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl.

In some embodiments, the methods include adjusting the pH and/or conductivity of the retentate fraction or the pulse protein isolate in two or more pH adjustment steps. In some cases, the pH is adjusted to a first pH range of from about 4.0 to about 6.6. Next, a second pH adjustment is made in which the pH of the retentate fraction or the pulse protein isolate is adjusted to be different, that is higher or lower, than the first pH of the retentate fraction or the pulse protein isolate. In some embodiments, the first pH adjustment is made to a pH of 4.0 to 6.0. In some embodiments, the pH achieved in the second pH adjustment is between 5.0 and 6.6. In various embodiments, the first pH is adjusted to 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. In various embodiments, the second pH is adjusted to 5.0, 5.1, 5.2, 5.3, 5.4, 5.5, 5.6, 5.7, 5.8, 5.9, 6.0, 6.1, 6.2, 6.3, 6.4, 6.5 or 6.6. In various embodiments, the conductivity is adjusted to 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mS/cm. In various embodiments, the salt used to modify the conductivity can be selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl.

### Pasteurization and Drying

In some embodiments, the methods include heating the retentate fraction or the pulse protein isolate in a pasteurization process and/or drying the retentate fraction or the pulse protein isolate. In some embodiments, the retentate fraction or the pulse protein isolate is heated to a temperature of from about 70° C. to about 80° C. for a period of time (e.g., 20-30 seconds) to kill pathogens (e.g., bacteria). In a particular embodiment, pasteurization is performed at 74° C. for 20 to 23 seconds. In particular embodiments where a dry pulse protein isolate is desired, the pulse protein isolate may be passed through a spray dryer to remove any residual water content. The typical spray drying conditions include an inlet temperature of 170° C. and an outlet temperature of 70° C. The final dried protein isolate powder may comprise less than 10% or less than 5% moisture content.

### Order of Steps and Additional Steps

It is to be understood that the steps of the methods discussed above or herein may be performed in alternative orders consistent with the objective of producing a pulse protein isolate. In some embodiments, the methods may include additional steps, such as for example: recovering the purified protein isolate (e.g., using centrifugation), washing the purified protein isolate, making a paste using the purified protein isolate, or making a powder using the purified protein isolate. In some embodiments, the purified protein isolate is rehydrated (e.g., to about 80% moisture content), and the pH of the rehydrated purified protein isolate is adjusted to a pH of about 6. Unless otherwise noted, none of the embodiments discussed herein include isoelectric precipitation of the pulse proteins from a protein rich fraction (e.g., at a pH of from about 5 to about 6).

### Pulse Protein Isolates

The present disclosure includes pulse protein isolates (e.g., mung bean protein isolates), including those prepared by the methods discussed above. The pulse protein isolates are edible and comprise one or more desirable food qualities, including but limited to, high protein content, high protein purity, reduced retention of small molecular weight non-protein species (including mono and disaccharides), reduced retention of oils and lipids, superior structure building properties such as high gel strength and gel elasticity, superior sensory properties, and selective enrichment of highly functional 8s globulin/beta conglycinin proteins.

In various embodiments, the pulse protein isolates provided herein are derived from dry beans, lentils, faba beans, dry peas, chickpeas, cowpeas, bambara beans, pigeon peas, lupins, vetches, adzuki, common beans, fenugreek, long beans, lima beans, runner beans, or tepary beans, soy beans, or mucuna beans. In various embodiments, the pulse protein isolates provided herein are derived from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse protein isolates are derived from mung beans. In some embodiments, the mung bean is Vigna radiata. In other embodiments, the milled composition may comprise almonds and other nuts, seeds such as sesame seeds, sunflower seeds, and other commonly consumed nuts, fruits and seeds. In various embodiments, the pulse protein isolate (e.g., mung bean protein isolate) discussed herein can be produced from any source of pulse protein (e.g., mung bean protein, including any varietal or cultivar of V. radiata). For example, the protein isolate can be prepared directly from whole plant material such as whole mung bean, or from an intermediate material made from the plant, for example, a dehulled bean, a flour, an air classified flour, a powder, a meal, ground grains, a cake (such as, for example, a defatted or de-oiled cake), or any other intermediate material suitable to the processing techniques disclosed herein to produce a pulse protein isolate. In some embodiments, the source of the plant protein may be a mixture of two or more intermediate materials. The examples of intermediate materials provided herein are not intended to be limiting.

### Characteristics of the Pulse Protein Isolates

In various embodiments, the pulse protein isolate (e.g., mung bean protein isolate) comprises pulse protein of from 50% to 60%, from 60% to 70%, from 70% to 80%, from 80% to 90%, or more. In some embodiments, the pulse protein isolate comprises 80%, 81%, 82%, 83%, 84%, 85%, 86%, 87%, 88%, 89%, 90%, 91%, 92%, 93%, 94%, 95%, 96%, 97%, 98%, or 99% or more pulse proteins. In some embodiments, at least 60% by weight of the pulse protein isolate is comprised of pulse proteins. In some embodiments, at least 65%, 70%, 75%, 80%, 85%, 90%, or 95% or more by weight of the pulse protein isolate comprises pulse proteins.

In some embodiments in which the pulse protein is mung bean protein, at least about 10%, 15%, 20%, 25%, 30%, 35%, 40%, 45%, 50%, 55%, 60%, 65%, 70%, 75%, 80%, 85% or greater than 85% by weight of the mung bean protein isolate consists of or comprises mung bean 8s globulin/beta-conglycinin. In other embodiments, about 60% to 80%, 65% to 85%, 70% to 90%, or 75% to 95% by weight of the mung bean protein isolate consists of or comprises mung bean 8s globulin/beta-conglycinin. In some embodiments, the mung bean protein isolate is reduced in the amount of 11s globulin relative to whole mung bean or mung bean flour. In some embodiments, the amount of 11s globulin is less than 10%, 8%, 8%, 7%, 6%, 5%, 4%, 3%, 2%, or 1% of the protein in the mung bean protein isolate.

In some embodiments, the pulse protein isolate (e.g., mung bean protein isolate) comprises about 1% to 10%, 2% to 9%, 3% to 8%, or 4% to 6% of carbohydrates (e.g., starch, polysaccharides, fiber) derived from the plant source of the isolate. In some embodiments, the pulse protein isolate comprises less than about 10%, 9%, 8%, 7%, 6% or 5% of carbohydrates derived from the plant source of the isolate. In some embodiments, the pulse protein isolate comprises about 9%, 8%, 7%, 6%, 5%, 4%, 3%, 2%, or about 1% of carbohydrates derived from the plant source of the isolate.

In some embodiments, the pulse protein isolate (e.g., mung bean protein isolate) comprises about 1% to 10%, 2% to 9%, 3% to 8%, or 4% to 6% of ash derived from the plant source of the isolate. In some embodiments, the pulse protein isolate comprises less than about 10%, 9%, 8%, 7%, 6% or 5% of ash derived from the plant source of the isolate. In some embodiments, the pulse protein isolate comprises about 9%, 8%, 7%, 6%, 5%, 4%, 3%, 2%, or about 1% of ash derived from the plant source of the isolate.

In some embodiments, the pulse protein isolate (e.g., mung bean protein isolate) comprises about 1% to 10%, 2% to 9%, 3% to 8%, or 4% to 6% of fats derived from the plant source of the isolate. In some embodiments, the pulse protein isolate comprises less than about 10%, 9%, 8%, 7%, 6% or 5% of fats derived from the plant source of the isolate. In some embodiments, the pulse protein isolate comprises about 9%, 8%, 7%, 6%, 5%, 4%, 3%, 2%, or about 1% of fats derived from the plant source of the isolate.

In some embodiments, the pulse protein isolate (e.g., mung bean protein isolate) comprises about 1% to 10% of moisture derived from the plant source of the isolate. In some embodiments, the pulse protein isolate comprises less than about 10%, 9%, 8%, 7%, 6% or 5% of moisture derived from the plant source of the isolate. In some embodiments, the pulse protein isolate comprises about 9%, 8%, 7%, 6%, 5%, 4%, 3%, 2%, or about 1% of moisture derived from the plant source of the isolate.

In various embodiments, the pulse protein isolate (e.g., mung bean protein isolate) has a density of less than 0.3 g/ml.

In various embodiments, the pulse protein isolate (e.g., mung bean protein isolate) comprises pulse proteins that, when formulated into a homogenized protein dispersion consisting of 12% w/w pulse protein, 0.35% w/w NaCl, and water has a separation ratio of less than 30% after 48 hours of storage at 4° C. In some embodiments, the pulse protein isolate (e.g., mung bean protein isolate) comprises pulse proteins that, when formulated into a homogenized protein dispersion consisting of 12% w/w pulse protein, 0.35% w/w NaCl, and water has a separation ratio of less than 25% after 48 hours of storage at 4° C. In some embodiments, the pulse protein isolate (e.g., mung bean protein isolate) comprises pulse proteins that, when formulated into a homogenized protein dispersion consisting of 12% w/w pulse protein, 0.35% w/w NaCl, and water has a separation ratio of less than 20% after 48 hours of storage at 4° C.

In various embodiments, the pulse protein isolate (e.g., mung bean protein isolate) has a storage modulus of from 25 Pa to 500 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s. In various embodiments, the pulse protein isolate (e.g., mung bean protein isolate) has a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s.

In various embodiments, the pulse protein isolate (e.g., mung bean protein isolate) has a linear viscoelastic region of from 25 Pa to 1500 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In various embodiments, the pulse protein isolate (e.g., mung bean protein isolate) has a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In some embodiments, the pulse protein isolate (e.g., mung bean protein isolate) has a linear viscoelastic region of less than 500 Pa at up to 10% strain, or a linear viscoelastic region of less than 200 Pa at up to 10% strain.

In various embodiments, the pulse protein isolate (e.g., mung bean protein isolate) comprises pulse proteins having a molecular size of less than 100 kilodaltons (kDa). In some embodiments, the pulse protein isolate comprises pulse proteins having a molecular size of less than 95 kDa, 90 kDa, 85 kDa, 80 kDa, 75 kDa, 70 kDa, 65 kDa, 60, kDa, 55 kDa, 50 kDa, 45 kDa, 40 kDa, 35 kDa, 30 kDa, 25 kDa, 20 kDa or 15 kDa. In various embodiments, the pulse protein isolate comprises pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the pulse protein isolate (e.g., mung bean protein isolate) comprises pulse proteins enriched in proteins having a molecular size of greater than 5 kilodaltons (kDa). In some embodiments, the pulse protein isolate comprises pulse proteins enriched in proteins having a molecular size of greater than 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa or 95 kDa. In some embodiments, the pulse protein isolated comprises pulse proteins enriched in proteins having a molecular size of less than 100 kDa. In some embodiments, the pulse protein isolate (e.g., mung bean protein isolate) comprises pulse proteins enriched in proteins having a molecular size of from 1 kDa to 99 kDa, from 1 kDa to 75 kDa, from 1 kDa to 50 kDa, from 1 kDa to 25 kDa, from 5 kDa to 99 kDa, from 5 kDa to 75 kDa, from 5 kDa to 50 kDa, from 5 kDa to 25 kDa, from 10 kDa to 99 kDa, from 10 kDa to 75 kDa, from 10 kDa to 50 kDa, or from 10 kDa to 25 kDa.

In various embodiments, the pulse protein isolate comprises, or is enriched in, pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the pulse protein isolate (e.g., mung bean protein isolate) comprises pulse proteins depleted in proteins having a molecular size of less than 5 kilodaltons (kDa). In some embodiments, the pulse protein isolate comprises pulse proteins depleted in proteins having a molecular size of less than 10 kDa, 15 kDa, 20 kDa, 25 kDa, 30 kDa, 40 kDa, 45 kDa, 50 kDa, 55 kDa, 60 kDa, 65 kDa, 70 kDa, 80 kDa, 85 kDa, 95 kDa or 95 kDa. In various embodiments, the pulse protein isolate comprises, or is enriched in, pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the pulse protein isolate may include particles of less than 1000, 900, 800, 700, 600, 500, 400, 300, 200 or 100 μm in size. In some embodiments, the pulse protein isolates may include particles less than 90, 80, 70, 60, 50, 45, 40, 35, 30, 25, or 20 μm in size.

In various embodiments, the pulse protein isolate may have a moisture content ranging from 5% to 90% or more. In some cases, the moisture content is 5% to 50%. In some cases, the moisture content is from 50% to 90%. In various embodiments, the moisture content is 10%, 20%, 30%, 40%, 50%, 60%, 70%, 80% or 90%.

### Reduced Allergen, Anti-Nutritional, and Environmental Contaminant Content

In some embodiments, the pulse protein isolates (e.g., mung bean protein isolate) provided herein have a reduced allergen content. In some embodiments, the reduced allergen content is relative to the allergen content of the plant source of the isolate. The pulse protein isolate or a composition comprising the pulse protein isolate may be animal-free, dairy-free, soy-free and gluten-free. Adverse immune responses such as hives or rash, swelling, wheezing, stomach pain, cramps, diarrhea, vomiting, dizziness and even anaphylaxis presented in subjects who are typically allergic to eggs may be averted. Further, the pulse protein isolate or a composition comprising the pulse protein isolate may not trigger allergic reactions in subjects based on milk, eggs, soy and wheat allergens. Accordingly, in some embodiments, the pulse protein isolate or a composition comprising the pulse protein isolate is substantially free of allergens.

Dietary anti-nutritional factors are chemical substances that can adversely impact the digestibility of protein, bioavailability of amino acids and protein quality of foods (Gilani et al., 2012). In some embodiments, the pulse protein isolates (e.g., mung bean protein isolates) provided herein have reduced amounts of anti-nutritional factors. In some embodiments, the reduced amount of anti-nutritional factors is relative to the content of the plant source of the isolate. In some embodiments, the reduced anti-nutritional factor is selected from the group consisting of tannins, phytic acid, hemagglutinins (lectins), polyphenols, trypsin inhibitors, α-amylase inhibitors, lectins and protease inhibitors.

In various embodiments, environmental contaminants are either free from the pulse protein isolates (e.g., mung bean protein isolates), below the level of detection of 0.1 ppm, or present at levels that pose no toxicological significance. In some embodiments, the reduced environmental contaminant is a pesticide residue. In some embodiments, the pesticide residue is selected from the group consisting of: chlorinated pesticides, including alachlor, aldrin, alpha-BHC, alpha-chlordane, beta-BHC, DDD, DDE, DDT, delta-BHC, dieldrin, endosulfan I, endosulfan II, endosulfan sulfate, endrin, endrin aldehyde, gamma-BHC, gamma-chlordane, heptachlor, heptachlor epoxide, methoxyclor, and permethrin; and organophosphate pesticides including azinophos methyl, carbophenothion, chlorfenvinphos, chlorpyrifos methyl, diazinon, dichlorvos, dursban, dyfonate, ethion, fenitrothion, malathion, methidathion, methyl parathion, parathion, phosalone, and pirimiphos methyl. In some embodiments, the reduced environmental contaminant is selected from residues of dioxins and polychlorinated biphenyls (PCBs), or mycotoxins such as aflatoxin B1, B2, G1, G2, and ochratoxin A.

### Other Food Functionality Characteristics of the Pulse Protein Isolates

In various embodiments, the pulse protein isolates (e.g., mung bean protein isolates) exhibit desirable functional characteristics such as emulsification, water binding, foaming and gelation properties comparable to an egg. In various embodiments, the pulse protein isolates exhibit one or more functional properties advantageous for use in food compositions. The functional properties may include, but are not limited to, crumb density, structure/texture, elasticity/springiness, coagulation, binding, moisturizing, mouthfeel, leavening, aeration/foaming, creaminess, and emulsification of the food composition. Mouthfeel is a concept used in the testing and description of food products. Products made using pulse protein isolates discussed herein can be assessed for mouthfeel. Products, e.g., baked goods, made using the pulse protein isolates have mouthfeel that is similar to products made with natural eggs. In some embodiments the mouthfeel of the products made using the pulse protein isolates is superior to the mouthfeel of previously known or attempted egg substitutes, e.g., bananas, modified whey proteins, or Egg Beaters™.

Examples of properties which may be included in a measure of mouthfeel include: Cohesiveness: degree to which the sample deforms before rupturing when biting with molars; Density: compactness of cross section of the sample after biting completely through with the molars; Dryness: degree to which the sample feels dry in the mouth; Fracturability: force with which the sample crumbles, cracks or shatters (fracturability encompasses crumbliness, crispiness, crunchiness and brittleness); Graininess: degree to which a sample contains small grainy particles, may be seen as the opposite of smoothness; Gumminess: energy required to disintegrate a semi-solid food to a state ready for swallowing; Hardness: force required to deform the product to given distance, i.e., force to compress between molars, bite through with incisors, compress between tongue and palate; Heaviness: weight of product perceived when first placed on tongue; Moisture absorption: amount of saliva absorbed by product; Moisture release: amount of wetness/juiciness released from sample; Mouthcoating: type and degree of coating in the mouth after mastication (for example, fat/oil); Roughness: degree of abrasiveness of product's surface perceived by the tongue; Slipperiness: degree to which the product slides over the tongue; Smoothness: absence of any particles, lumps, bumps, etc., in the product; Uniformity: degree to which the sample is even throughout; homogeneity; Uniformity of Bite: evenness of force through bite; Uniformity of Chew: degree to which the chewing characteristics of the product are even throughout mastication; Viscosity: force required to draw a liquid from a spoon over the tongue; and Wetness: amount of moisture perceived on product's surface.

The pulse protein isolates discussed herein may also have one or more functional properties alone or when incorporated into a food composition. Such functional properties may include, but are not limited to, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, at least one functional property of the pulse protein isolate differs from the corresponding functional property of the source of the plant protein. In some embodiments, at least one functional property of the pulse protein isolate (alone or when incorporated into a food composition) is similar or equivalent to the corresponding functional property of a reference food product, such as, for example, an egg (liquid, scrambled, or in patty form), a cake (e.g., pound cake, yellow cake, or angel food cake), a cream cheese, a pasta, an emulsion, a confection, an ice cream, a custard, milk, a deli meat, chicken (e.g., chicken nuggets), or a coating. In some embodiments, the pulse protein isolate, either alone or when incorporated into a composition, is capable of forming a gel under heat or at room temperature.

### Modified Organoleptic Properties

The pulse protein isolates discussed herein may have modulated organoleptic properties of one or more of the following characteristics: astringent, beany, bitter, burnt, buttery, nutty, sweet, sour, fruity, floral, woody, earthy, beany, spicy, metallic, sweet, musty, grassy, green, oily, vinegary, neutral and bland flavor or aromas. In some embodiments, the pulse protein isolates exhibit modulated organoleptic properties such as a reduction or absence in one or more of the following: astringent, beany, bitter, burnt, buttery, nutty, sweet, sour, fruity, floral, woody, earthy, beany, spicy, metallic, sweet, musty, grassy, green, oily, vinegary neutral and bland flavor or aromas.

In some cases, methods to reduce or remove at least one impurity that may impart or is associated with an off-flavor or off-odor in the pulse protein isolate may be undertaken. The one or more impurity may be a volatile or nonvolatile compound and may comprise, for example, lipoxygenase, which is known to catalyze oxidation of fatty acids. In other cases, the at least one impurity may comprise a phenol, an alcohol, an aldehyde, a sulfide, a peroxide, or a terpene. Other biologically active proteins classified as albumins may also be removed, including lectins and protease inhibitors such as serine protease inhibitors and tryptic inhibitors. In some embodiments, impurities are reduced by a solid absorption procedure using, for example, charcoal, a bentonite clay, or activated carbon.

In some embodiments, the at least one impurity may comprise one or more substrates for an oxidative enzymatic activity, for example one or more fatty acids. In some embodiments, the pulse protein isolates contain reduced amounts of one or more fatty acids selected from: C14:0 (methyl myristate); C15:0 (methyl pentadecanoate); C16:0 (methyl palmitate; C16:1 methyl palmitoleate; C17:0 methyl heptadecanoate; C18:0 methyl stearate; C18:1 methyl oleate; C18:2 methyl linoleate; C18:3 methyl alpha linoleate; C20:0 methyl eicosanoate; and C22:0 methyl behenate to reduce rancidity.

In some embodiments, the pulse protein isolate (e.g., mung bean protein isolate) has a reduced oxidative enzymatic activity relative to the source of the pulse protein. For example, a purified mung bean isolate can have about a 5%, 10%, 15%, 20%, or 25% reduction in oxidative enzymatic activity relative to the source of the mung bean protein. In some embodiments, the oxidative enzymatic activity is lipoxygenase activity. In some embodiments, the pulse protein isolate has lower oxidation of lipids or residual lipids relative to the source of the plant protein due to reduced lipoxygenase activity.

In additional embodiments, reducing the at least one impurity comprises removing a fibrous solid, a salt, or a carbohydrate. Reducing such impurity comprises removing at least one compound that may impart or is associated with the off-flavor or off-odor. Such compounds may be removed, for example, using an activated charcoal, carbon, or clay. As another example, the at least one compound may be removed using a chelating agent (e.g., EDTA, citric acid, or a phosphate) to inhibit at least one enzyme that oxidizes a lipid or a residual lipid. In a particular example, EDTA may be used to bind co-factor for lipoxygenase, an enzyme that can oxidize residual lipid to compounds, e.g. hexanal, which are known to leave to off-flavors.

### Food Compositions Containing Pulse Protein Isolates

The pulse protein isolates (e.g., mung bean protein isolates) discussed herein may be incorporated into a food composition along with one or more other edible ingredients. In some cases, the pulse protein isolate may be used as a direct protein replacement of animal- or vegetable-based protein in a variety of conventional food and beverage products across multiple categories. In some embodiments, the use levels range from 3 to 90% w/w of the final product. Exemplary food compositions in which the pulse protein isolates can be used are discussed below. In some embodiments, the pulse protein isolate is used as a supplement to existing protein in food products. In any of the various embodiments of the food compositions, the pulse protein isolate may be contacted with a cross-linking enzyme to cross-link the pulse proteins. In various embodiments, the cross-linking enzyme is selected from transglutaminase, sortase, subtilisin, tyrosinase, laccase, peroxidase, or lysyl oxidase. In some embodiments, the cross-linking enzyme is transglutaminase. In any of the various embodiments of the food compositions, the pulse protein isolate may be contacted with a protein modifying enzyme such as papain, pepsin, rennet, coagulating enzymes or sulfhydryl oxidase to modify the structure of the pulse proteins.

The pulse protein isolates provided herein are suitable for various food applications and can be incorporated into, e.g., edible egg-free emulsion, egg analog, egg-free scrambled eggs, egg-free patty, egg-free pound cake, egg-free angel food cake, egg-free yellow cake, egg-free cream cheese, egg-free pasta dough, egg-free custard, egg-free ice cream, and dairy-free milk. The pulse protein isolates can also be used as replacement ingredients in various food applications including but not limited to meat substitutes, egg substitutes, baked goods and fortified drinks

In various embodiments, one or more pulse protein isolates can be incorporated into multiple food compositions, including liquid and patty scrambled egg substitutes to a desired level of emulsification, water binding and gelation. In an embodiment, a functional egg replacement product comprises pulse protein isolate (8-15%), and one or more of: oil (10%), hydrocolloid, preservative, and optionally flavors, water, lecithin, xanthan, sodium carbonate, and black salt.

In some embodiments, the pulse protein isolate is incorporated in an egg substitute composition. In some such embodiments, the organoleptic property of the pulse protein isolate (e.g., a flavor or an aroma) is similar or equivalent to a corresponding organoleptic property of an egg. The egg substitute composition may exhibit at least one functional property (e.g., emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color) that is similar or equivalent to a corresponding functional property of an egg. In addition to the pulse protein isolate, the egg substitute composition may include one or more of iota-carrageenan, gum arabic, konjac, xanthan gum, or gellan.

In some embodiments, the pulse protein isolate is incorporated in an egg-free cake, such as a pound cake, a yellow cake, or an angel food cake. In some such embodiments, at least one organoleptic property (e.g., a flavor or an aroma) of the egg-free cake is similar or equivalent to a corresponding organoleptic property of a cake containing eggs. The egg-free cake may exhibit at least one functional property similar or equivalent to a corresponding functional property of a cake containing eggs. The at least one function property may be, for example, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments in which the pulse protein isolate is included in an egg-free pound cake, a peak height of the egg-free pound cake is at least 90% of the peak height of a pound cake containing eggs.

In some embodiments, the pulse protein isolate is incorporated into an egg-free cake mix or an egg-free cake batter. In some such embodiments, the egg-free cake mix or batter has at least one organoleptic property (e.g., a flavor or aroma) that is similar or equivalent to a corresponding organoleptic property of a cake mix or batter containing eggs. The egg-free cake mix or batter may exhibit at least one functional property similar or equivalent to a corresponding functional property of a cake batter containing eggs. The at least one functional property may be, for example, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments in which the pulse protein isolate is included in an egg-free pound cake batter, a specific gravity of the egg-free pound cake batter is 0.95-0.99.

In some cases, increased functionality is associated with the pulse protein isolate in a food composition. For instance, food products produced with the pulse protein isolates discussed herein may exhibit increased functionality in dome or crack, cake resilience, cake cohesiveness, cake springiness, cake peak height, specific gravity of batter, center doming, center crack, browning, mouthfeel, spring-back, off flavors or flavor.

In some embodiments, the pulse protein isolate is included in a cream cheese, a pasta dough, a pasta, a milk, a custard, a frozen dessert (e.g., a frozen dessert comprising ice cream), a deli meat, or chicken (e.g., chicken nuggets).

In some embodiments, the pulse protein isolate is incorporated into a food or beverage composition, such as, for example, an egg substitute, a cake (e.g., a pound cake, a yellow cake, or an angel food cake), a cake batter, a cake mix, a cream cheese, a pasta dough, a pasta, a custard, an ice cream, a milk, a deli meat, or a confection. The food or beverage composition may provide sensory impressions similar or equivalent to the texture and mouthfeel that replicates a reference food or beverage composition. In some embodiments, before being included in a food or beverage composition, the pulse protein isolate is further processed in a manner that depends on a target application for the pulse protein isolate. For example, the pulse protein isolate may be diluted in a buffer to adjust the pH to a pH appropriate for the target application. As another example, the pulse protein isolate may be concentrated for use in the target application. As yet another example, the pulse protein isolate may be dried for use in the target application. Various examples of food compositions comprising the pulse protein isolates discussed herein are provided below.

### Scrambled Egg Analog Using Transglutaminase

In some embodiments, the pulse protein isolates are incorporated into a scrambled egg analog in which the pulse protein isolate (e.g., mung bean protein isolate) has been contacted with transglutaminase (or other cross-linking enzyme) to provide advantageous textural, functional and organoleptic properties. Food processing methods employing transglutaminases are known in the art.

In some embodiments, the transglutaminase is microencapsulated when utilized in the egg analogs provided herein. Microencapsulation of transglutaminase enzyme in such egg mimetic emulsions maintains a stable emulsion by preventing contact of the protein substrate with the transglutaminase enzyme. A cross-linking reaction is initiated upon heating to melt the microencapsulating composition. In some embodiments, the transglutaminase is immobilized on inert porous beads or polymer sheets, and contacted with the egg mimetic emulsions.

In certain aspects of the invention, the method for producing an egg substitute composition comprises contacting a pulse protein isolate with an amount of transglutaminase, preferably between 0.0001% to 0.1%. In some embodiments, the method provides an amount of transglutaminase between 0.001% and 0.05%. In some embodiments, the method provides an amount of transglutaminase between 0.001% and 0.0125%.

In various embodiments, the scrambled egg analog comprises a pulse protein isolate described herein, along with one or more of the following components: water, disodium phosphate and oil. In some embodiments, the scrambled egg analog further comprises NaCl. In some embodiments, the scrambled egg analog has been contacted with transglutaminase. In a particular embodiment, the scrambled egg analog comprises: Protein Solids: 11.3 g, Water: 81.79 g, Disodium phosphate: 0.4 g, Oil: 6.2 g, NaCl: 0.31 g (based on total weight of 100 g) wherein the protein solids are contacted with between 0.001% and 0.0125% of transglutaminase.

In some embodiments, the composition lacks lipoxygenase.

### Vegan Patty

Pulse protein isolates (e.g., mung bean protein isolates) can be used as the sole gelling agent in a formulated vegan patty. In some embodiments, a hydrocolloid system comprised of iota-carrageenan and gum arabic enhances native gelling properties of the pulse protein isolate in a formulated patty. In other embodiments, a hydrocolloid system comprised of high-acyl and low-acyl gellan in a 1.5:1 ratio enhances native gelling properties of the pulse protein isolate in a formulated patty. In further embodiments, a hydrocolloid system comprised of konjac and xanthan gum enhances native gelling properties of the pulse protein isolate in a formulated patty.

### Egg-Free Emulsion

In another embodiment, pulse protein isolates (e.g., mung bean protein isolates) are included in an edible egg-free emulsion. In some embodiments, the emulsion comprises one or more additional components selected from water, oil, fat, hydrocolloid, and starch. In some embodiments, at least or about 60-85% of the edible egg-free emulsion is water. In some embodiments, at least or about 10-20% of the edible egg-free emulsion is the pulse protein isolate. In some embodiments, at least or about 5-15% of the edible egg-free emulsion is oil or fat. In some embodiments, at least or about 0.01-6% of the edible egg-free emulsion is the hydrocolloid fraction or starch. In some embodiments, the hydrocolloid fraction comprises high-acyl gellan gum, low-acyl gellan gum, iota-carrageenan, gum arabic, konjac, locust bean gum, guar gum, xanthan gum, or a combination of one or more gums thereof. In some embodiments, the emulsion further comprises one or more of: a flavoring, a coloring agent, an antimicrobial, a leavening agent, and salt. In some embodiments, the emulsion further comprises phosphate.

In an embodiment, the edible egg-free emulsion has a pH of about 5.6 to 6.8. In some cases, the edible egg-free emulsion comprises water, a pulse protein isolate as described herein, an enzyme that modifies a structure of the protein isolate, and oil or fat. In some embodiments, the enzyme comprises a transglutaminase or proteolytic enzyme. In some embodiments, at least or about 70-85% of the edible egg-free emulsion is water. In some embodiments, at least or about 7-15% of the edible egg-free emulsion is the pulse protein isolate. In some embodiments, at least or about 0.0005-0.0025% (5-25 parts per million) of the edible egg-free emulsion is the enzyme that modifies the structure of the pulse protein isolate. In some embodiments, at least or about 5-15% of the edible egg-free emulsion is oil or fat.

### Baked Cake Mixes and Batters

In another embodiment, pulse protein isolates (e.g., mung bean protein isolates) are included in one or more egg-free cake mixes, suitable for preparing one or more egg-free cake batters, from which one or more egg-free cakes can be made. In some embodiments, the egg-free cake mix comprises flour, sugar, and a pulse protein isolate. In some embodiments, the egg-free cake mix further comprises one or more additional components selected from: cream of tartar, disodium phosphate, baking soda, and a pH stabilizing agent. In some embodiments, the flour comprises cake flour.

In another embodiment, pulse protein isolates (e.g., mung bean protein isolates) are included in an egg-free cake batter comprising an egg-free cake mix described above, and water. In some embodiments, the egg-free cake batter is an egg-free pound cake batter, an egg-free angel food cake batter, or an egg-free yellow cake batter. In some embodiments, the egg-free cake batter has a specific gravity of 0.95-0.99.

In an embodiment, an egg-free pound cake mix comprises flour, sugar, and a pulse protein isolate. In some embodiments, the flour comprises cake flour. In some embodiments, the egg-free pound cake mix further comprises oil or fat. In some embodiments, the oil or fat comprises butter or shortening. In some embodiments, at least or about 25-31% of the egg-free pound cake batter is flour. In some embodiments, at least or about 25-31% of the egg-free pound cake batter is oil or fat. In some embodiments, at least or about 25-31% of the egg-free pound cake batter is sugar. In some embodiments, at least or about 6-12% of the egg-free pound cake batter is the pulse protein isolate. In some embodiments, the batter further comprises disodium phosphate or baking soda.

In an embodiment, an egg-free pound cake batter comprises an egg-free pound cake mix described above, and further comprises water. In some embodiments, the egg-free pound cake batter comprises about four parts of the egg-free pound cake mix; and about one part water. In some embodiments, at least or about 20-25% of the egg-free pound cake batter is flour. In some embodiments, at least or about 20-25% of the egg-free pound cake batter is oil or fat. In some embodiments, at least or about 20-25% of the egg-free pound cake batter is sugar. In some embodiments, at least or about 5-8% of the egg-free pound cake batter is the pulse protein isolate. In some embodiments, at least or about 18-20% of the egg-free pound cake batter is water.

In an embodiment, an egg-free angel food cake mix comprises flour, sugar, and a pulse protein isolate. In some embodiments, at least or about 8-16% of the egg-free angel food cake mix is flour. In some embodiments, at least or about 29-42% of the egg-free angel food cake mix is sugar. In some embodiments, at least or about 7-10% of the egg-free angel food cake mix is the pulse protein isolate. In some embodiments, the egg-free angel food cake mix further comprises cream of tartar, disodium phosphate, baking soda, or a pH stabilizing agent. In some embodiments, the flour comprises cake flour. Also provided herein is an egg-free angel food cake batter comprising an egg-free angel food cake mix described above, and water.

In an embodiment, an egg-free yellow cake mix comprises flour, sugar, and a pulse protein isolate. In some embodiments, at least or about 20-33% of the egg-free yellow cake mix is flour. In some embodiments, at least or about 19-39% of the egg-free yellow cake mix is sugar. In some embodiments, at least or about 4-7% of the egg-free yellow cake mix is the pulse protein isolate. In some embodiments, the egg-free yellow cake mix further comprises one or more of baking powder, salt, dry milk, and shortening. Also provided herein is an egg-free yellow cake batter comprising an egg-free yellow cake mix described above, and water.

Sensory quality parameters of cakes made with the pulse protein isolates are characterized as fluffy, soft, airy texture. The peak height is measured to be 90-110% of pound cake containing eggs. The specific gravity of cake batter with the purified pulse protein isolate is 0.95-0.99, similar to that of cake batter with whole eggs of 0.95-0.96.

### Cream Cheese Analog

In another embodiment, pulse protein isolates (e.g., mung bean protein isolates) are included in an egg-free cream cheese. In some embodiments, the egg-free cream cheese comprises one or more additional components selected from water, oil or fat, and hydrocolloid. In some embodiments, at least or about 75-85% of the egg-free cream cheese is water. In some embodiments, at least or about 10-15% of the egg-free cream cheese is the pulse protein isolate. In some embodiments, at least or about 5-10% of the egg-free cream cheese is oil or fat. In some embodiments, at least or about 0.1-3% of the egg-free cream cheese is hydrocolloid. In some embodiments, the hydrocolloid comprises xanthan gum or a low-methoxy pectin and calcium chloride system. In some embodiments, the egg-free cream cheese further comprises a flavoring or salt. In some embodiments, one or more characteristics of the egg-free cream cheese is similar or equivalent to one or more corresponding characteristics of a cream cheese containing eggs. In some embodiments, the characteristic is a taste, a viscosity, a creaminess, a consistency, a smell, a spreadability, a color, a thermal stability, or a melting property. In some embodiments, the characteristic comprises a functional property or an organoleptic property. In some embodiments, the functional property comprises: emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, the organoleptic property comprises a flavor or an odor.

### Egg-Free Pasta Dough

In another embodiment, pulse protein isolates (e.g., mung bean protein isolates) are included in an egg-free pasta dough. In some embodiments, the egg-free pasta dough comprises one or more additional components selected from flour, oil or fat, and water. In some embodiments, the flour comprises semolina flour. In some embodiments, the oil or fat comprises extra virgin oil. In some embodiments, the egg-free pasta dough further comprises salt. Also provided herein is an egg-free pasta made from an egg-free pasta dough described above. In some embodiments, the egg-free pasta is fresh. In some embodiments, the egg-free pasta is dried. In some embodiments, one or more characteristics of the egg-free pasta is similar or equivalent to one or more corresponding characteristics of a pasta containing eggs. In some embodiments, the one or more characteristics comprise chewiness, density, taste, cooking time, shelf life, cohesiveness, or stickiness. In some embodiments, the one or more characteristics comprise a functional property or an organoleptic property. In some embodiments, the functional property comprises: emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, the organoleptic property comprises a flavor or an odor.

### Plant-Based Milk

In another embodiment, pulse protein isolates (e.g., mung bean protein isolates) are included in a plant-based milk. In some embodiments, the plant-based milk comprises one or more additional components selected from water, oil or fat, and sugar. In some embodiments, at least or about 5% of the plant-based milk is the pulse protein isolate. In some embodiments, at least or about 70% of the plant-based milk is water. In some embodiments, at least or about 2% of the plant-based milk is oil or fat. In some embodiments, the plant-based milk further comprises one or more of: disodium phosphate, soy lecithin, and trace minerals. In particular embodiments, the plant-based milk is lactose-free. In other particular embodiments, the plant-based milk does not comprise gums or stabilizers.

### Egg-Free Custard

In another embodiment, pulse protein isolates (e.g., mung bean protein isolates) are included in an egg-free custard. In some embodiments, the egg-free custard comprises one or more additional components selected from cream and sugar. In some embodiments, at least or about 5% of the egg-free custard is the pulse protein isolate. In some embodiments, at least or about 81% of the egg-free custard is cream. In some embodiments, at least or about 13% of the egg-free custard is sugar. In some embodiments, the egg-free custard further comprises one or more of: iota-carrageenan, kappa-carrageenan, vanilla, and salt. In some embodiments, the cream is heavy cream.

### Egg-Free Ice Cream

In another embodiment, pulse protein isolates (e.g., mung bean protein isolates) are included in an egg-free ice cream. In some embodiments, the egg-free ice cream is a soft-serve ice cream or a regular ice cream. In some embodiments, the egg-free ice cream comprises one or more additional components selected from cream, milk, and sugar. In some embodiments, at least or about 5% of the egg-free ice cream is the protein isolate. In some embodiments, at least or about 41% of the egg-free ice cream is cream. In some embodiments, at least or about 40% of the egg-free ice cream is milk. In some embodiments, at least or about 13% of the egg-free ice cream is sugar. In some embodiments, the egg-free ice cream further comprises one or more of iota carrageenan, kappa carrageenan, vanilla, and salt. In some embodiments, the cream is heavy cream. In some embodiments, the milk is whole milk. In particular embodiments, the egg-free ice cream is lactose-free. In some embodiments, the egg-free ice cream does not comprise gums or stabilizers. In some embodiments, the egg-free ice provides a traditional mouthfeel and texture of an egg-based ice cream but melts at a slower rate relative to an egg-based ice cream.

### Fat Reduction Shortening System (FRSS)

In another embodiment, pulse protein isolates (e.g., mung bean protein isolates) are included in a fat reduction shortening system. In some embodiments, the FRSS comprises one or more additional components selected from water, oil or fat. In some embodiments, the FRSS further comprises sodium citrate. In further some embodiments, the FRSS further comprises citrus fiber. In some embodiments, at least or about 5% of the FRSS is the pulse protein isolate. In preferred embodiments, the pulse protein-based FRSS enables a reduction in fat content in a food application (e.g., a baking application) utilizing the FRSS, when compared to the same food application utilizing an animal and/or dairy based shortening. In some embodiments, the reduction in fat is at least 10%, 20%, 30% or 40% when compared to the same food application utilizing an animal and/or dairy based shortening.

### Meat Analogues

In another embodiment, pulse protein isolates (e.g., mung bean protein isolates) are included in a meat analogue. In some embodiments, the meat analogue comprises one or more additional components selected from water, oil, disodium phosphate, transglutaminase, starch and salt. In some embodiments, at least or about 10% of the meat analogue is the pulse protein isolate. In some embodiments, preparation of the meat analogue comprises mixing the components of the meat analogue into an emulsion and pouring the emulsion into a casing that can be tied into a chubb. In some embodiments, chubs containing the meat analogue are incubated in a water bath at 50° C. for 2 hours. In further embodiments, the incubated chubbs are pressure cooked. In some embodiments, the pressure cooking occurs at 15 psi at about 121° C. for 30 minutes.

### Food Applications: Co-Ingredients

Various gums, phosphates, starches, preservatives, and other ingredients may be included in the food compositions comprising the pulse protein isolates.

Various gums useful for formulating one or more pulse protein based food products described herein include, e.g., konjac, gum acacia, Versawhip, Guar+Xanthan, Q-extract, CMC 6000 (Carboxymethylcellulose), Citri-Fi 200 (citrus fiber), Apple fiber, Fenugreek fiber.

Various phosphates useful for formulating one or more pulse protein based food products described herein include disodium phosphate (DSP), sodium hexamethaphosphate (SHMP), and tetrasodium pyrophosphate (TSPP).

Starch may be included as a food ingredient in the pulse protein food products described herein. Starch has been shown to have useful emulsifying properties; starch and starch granules are known to stabilize emulsions. Starches are produced from plant compositions, such as, for example, arrowroot starch, cornstarch, tapioca starch, mung bean starch, potato starch, sweet potato starch, rice starch, sago starch, wheat starch.

In certain embodiments, the food compositions comprise an effective amount of an added preservative in combination with the pulse protein isolate. The preservative may include ascorbic acid, citric acid, sodium benzoate, calcium propionate, sodium erythorbate, sodium nitrite, calcium sorbate, potassium sorbate, BHA, BHT, EDTA, tocopherols (Vitamin E) or antioxidants.

### Storage and Shelf Life of Food Compositions

In some embodiments, the food compositions comprising the pulse protein isolates may be stable in storage at room temperature for up to 1, 2, 3, 4, 5, 6, 7, 8, 9, or 10 weeks. In some embodiments, the food compositions comprising the pulse protein isolates may be stable for storage at room temperature for months, e.g., greater than 1, 2, 3, 4, 5, 6, 7, 8, 9, 10, 11, 12, or 13 months. In some embodiments, the food compositions comprising the pulse protein isolates may be stable for refrigerated or freezer storage for months, e.g., greater than 1, 2, 3, 4, 5, 6, 7, 8, 9, 10, 11, 12, or 13 months. In some embodiments, the food compositions comprising the pulse protein isolates may be stable for refrigerated or freezer storage for years, e.g., greater than 1, 2, 3, 4, 5, 6, 7, 8, 9, 10, 11, 12, or 13 years.

In some embodiments, storage as a dry material can increase the shelf-life of the pulse protein isolate or a food composition comprising the pulse protein isolate. In some embodiments, the pulse protein isolate or a food composition comprising the pulse protein isolate is stored as a dry material for later reconstitution with a liquid, e.g., water. In some embodiments, the pulse protein isolate or the food composition is in powdered form, which may be less expensive to ship, lowers risk for spoilage and increases shelf-life (due to greatly reduced water content and water activity).

In various embodiments, a food composition (e.g., an egg-free liquid egg analog product) comprising the pulse protein isolate has a viscosity of less than 500 cP after storage for thirty days at 4° C. In some cases, the composition has a viscosity of less than 500 cP after storage for sixty days at 4° C. In various embodiments, a food composition (e.g., an egg-free liquid egg analog product) comprising the pulse protein isolate has a viscosity of less than 450 cP after storage for thirty days at 4° C. In some cases, the composition has a viscosity of less than 450 cP after storage for sixty days at 4° C.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Ultrafiltration Process for Preparing Pulse Protein Isolates

The following example discusses an exemplary process for the production of an ultrafiltered (UF) pulse protein isolate, and also the production of an isoelectrically-precipitated (IEP) control sample for use as a comparator in following examples characterizing the properties of the UF pulse protein isolate.

Ultrafiltered Pulse Protein Isolate: 40 kg of Mung bean flour (102) that was preprocessed by drying and grinding was extracted (104) with 200 kg water, 600 g salt (NaCl), 100 mL antifoam in a Breddo liquefier (Corbion Inc). The mixing was performed for 2.5 minutes. The pH at the end of the run was adjusted to 7.0 using 1 M NaOH solution. The flour slurry (105) was then centrifuged to perform a starch solid separation (106) using a decanter (SG2-100, Alfalaval Inc). The major portion of the starch solids and unextracted material (decanter heavy phase) was separated from the liquid suspension (decanter light phase). The resuspension stream (light phase) was further clarified using a disc stack centrifuge (Clara 80, Alfalaval Inc.) into a high solids slurry (disc stack heavy phase) and a clarified resuspension (107—disc stack light phase). The disc stack heavy phase typically consists of fat, ash, starch and the protein carried over with the liquid portion of the slurry.

Half of the disc stack light phase (protein-rich fraction) was then processed through an ultrafiltration-diafiltration process (109) with a custom designed membrane purification unit (Alfalaval Inc.). This membrane unit was setup with a 10 kDa membrane from Alfalaval Inc. (3838RC10PP). The disc stack light phase was concentrated from 75 kg to about 20 kg (3-4× concentration). The concentrated protein suspension was further diafiltered with DI water in three steps adding about equal amount of water at each step as the concentrate weight. The stream (110) of diafiltered UF concentrate (19.5 kg) was then collected and the pH of this concentrate was adjusted (111) from 7 to 6.1 using 20% w/w citric acid solution. Salt (NaCl) was added to adjust the conductivity in the 2-3 mS/cm range and not modified. The mildly denatured protein concentrate material (112) was then heat treated (113) using a microthermics UHT unit with the pasteurization condition set at 72.5° C. and 30 sec hold time. The heat-treated material (114) was then spray dried (115) with a SPX Anhydro M400 spray dryer (GEA Niro Inc.) with the inlet temp at 180° C., outlet temp at 85° C. using a nozzle atomizer to obtain protein isolate (116). An illustration of this process, including the numbers (102-116) noted above, is shown in FIG. 1.

Isoelectrically-Precipitated Pulse Protein Isolate Control: The other half of the disc stack light phase was then transferred to the liquefier tank. The pH was adjusted to 5.6 with 20% w/w citric acid. The slurry was mixed and run through the decanter (SG2-100, Alfalaval Inc.) in recirc mode until the spin down on the decanter light phase was negligible. Then the decanter was shut down and the protein pellet collected on the decanter heavy phase side. The pellet was resuspended with 3.5× deionized water to get the concentration in the range to minimize spray drier losses. The resuspended protein solution was adjusted to a pH of 6 using 1M NaOH and salt was added to obtain the conductivity in the 2-3 mS/cm range. This material was then heat treated and spray dried to obtain an isoelectrically precipitated isolate for use as a control in Examples 3-6.

### Example 2: Evaluation of Extraction Parameters on Pulse Protein Recovery

The following example discusses the evaluation of various extraction parameters for effects on pulse protein recovery. The extraction of pulse proteins can be mass transfer limited or diffusion limited. Factors affecting mass transfer include mechanical factors such as temperature, solid:liquid ratio, and agitation. Factors affecting diffusion include the extraction buffer (pH, and salt concentration), and particle size. A typical extraction used in this example included 150 g ground heat treated flour (mung bean) mixed with 750 g water and 2.25 g salt (NaCl). Once homogenous, the pH of the mixture was adjusted to 7 using 1M NaOH solution. The pH adjusted flour slurry was centrifuged at 6000 G for 15 minutes in a bench top centrifuge (Lynx 6000, Thermofisher Scientific). The solid pellet was removed and the liquid was weighed, dried for total solids (by standard AOAC method—8 hours at 105° C.), protein analysis (Dumas method).

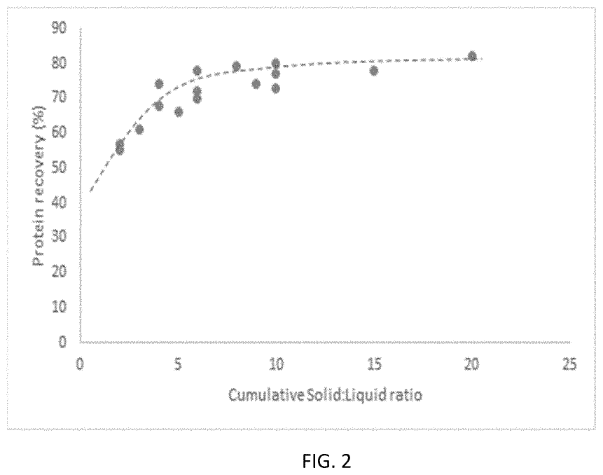

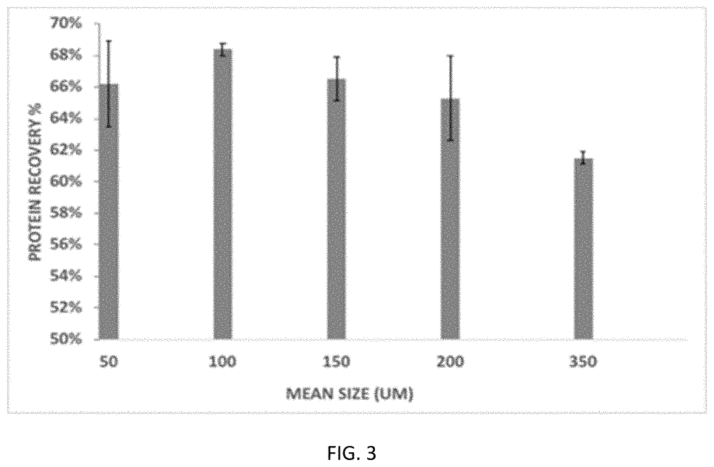

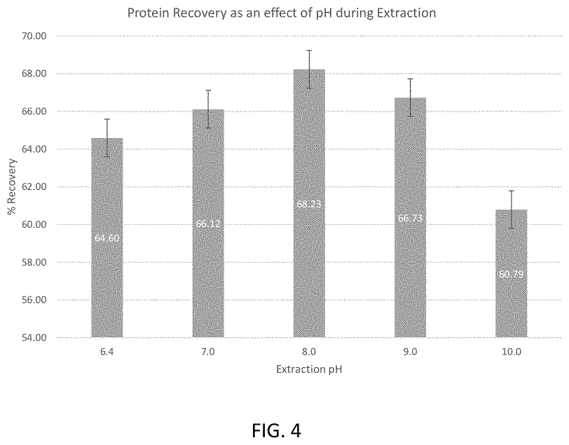

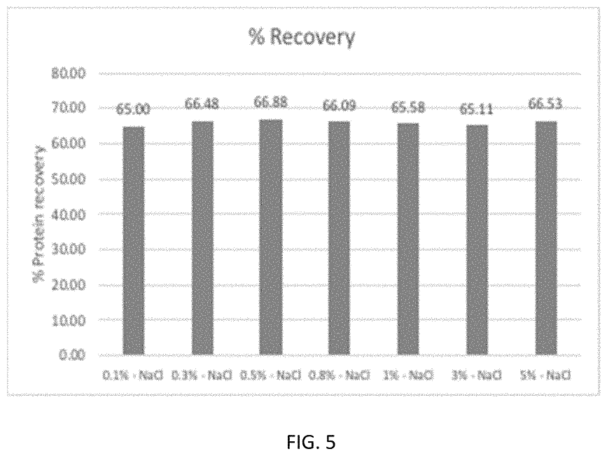

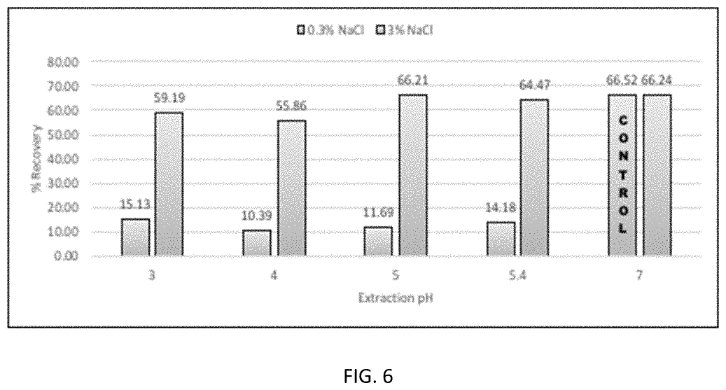

Effect of solid:liquid ratio on protein recovery: Three serial extractions were performed on the heavy-phase generated after each starch separation process and the protein yield was evaluated at each stage. The effects of solid:liquid ratio variation on protein recovery are shown in FIG. 2. As shown in FIG. 2, a solid:liquid ratio of about 1:6 yielded nearly the ˜80% maximum protein recovery while minimizing the liquid content for downstream processing.
Effect of particle size distribution on protein recovery: Protein extractions using the same starting bean milled at five different conditions to get a range of mean particle sizes were performed to determine the effects on protein recovery. Particles sizes for the five tested conditions were 50 μm, 100 μm, 150 μm, 200 μm, and 350 μm. As illustrated in FIG. 3, a particle size of from 50 μm to 200 μm yielded nearly equivalent extraction efficiency.
Effect of temperature on protein recovery: Temperatures ranging from 30° C. to 60° C. were evaluated for the effects on extraction protein recovery. There was no effect on protein recovery at the tested temperatures (data not shown).
Effect of extraction pH on protein recovery: Extraction at pH 6.4 (unadjusted), pH 7.0, pH 8.0, pH 9.0 and pH 10.0 was performed to assess the effect on protein recovery. Protein recovery was higher in the pH range 7-9, and pH 8 showed slightly higher recovery. Results are shown in FIG. 4.
Effect of NaCl concentration on protein recovery: The extraction process was performed at pH 7.0 with varying NaCl concentrations ranging from 0.1% to 5% w/v to evaluate the effects on protein recovery. There was no significant variation in protein recovery over the NaCl concentrations tested in the extraction process carried out at pH 7.0, as shown in FIG. 5. A similar result was achieved in the absence of salt (data not shown).
Combined effect of PH and NaCl concentration: The effects of pH and salt concentration on protein extraction recovery were studied. Two sets of experiments were performed, the first using 0.3% NaCl at a pH ranging from 3 to 7, and a second using 3% NaCl at a pH ranging from 3 to 7. The results are shown in FIG. 6. Protein recovery increased significantly at lower pH with 3% salt as compared to 0.3% salt.

### Example 3: Characterization of Density of Pulse Protein Isolates

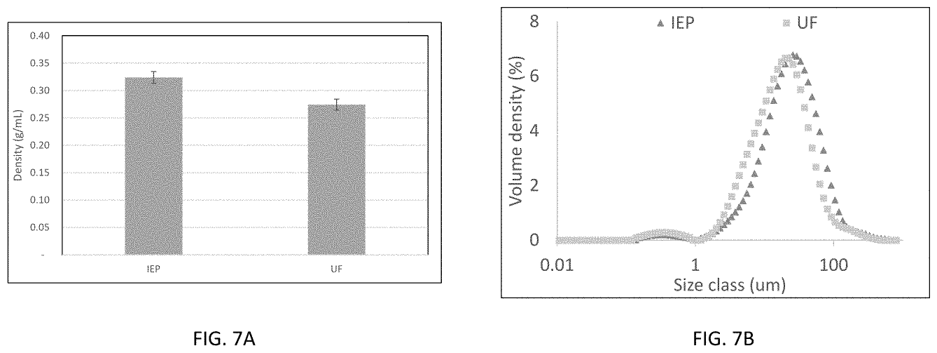

The density and particle size distribution of UF and IEP isolates prepared according the methods discussed in Example 1 were analyzed. Density measurements were performed by weighing out 5 g of IEP isolate and placing the isolate in a 100 mL graduated container. The container was tapped and the volume was noted. The process was repeated with 10 g, 15 g, and 20 g of the IEP isolate, and then with 5 g, 10 g, 15 g, and 20 g of the UF isolate. Density was calculated by dividing the weight by the volume. As shown in FIG. 7A, the IEP isolate was significantly denser than the UF isolate.

The density differences illustrated in FIG. 7A are not explained by a difference in particle size of the two isolates. As shown in FIG. 7B, the particle size distribution of the IEP and UF isolates showed no significant difference in particle sizes, with nearly overlapping size distributions as measured using the mastersizer aero 3000 (Malvern Inc.). If anything, based on the particle size distribution, one may have expected the density differences to be reversed, with the slightly larger particle sizes of the IEP isolate to yield a lower density.

### Example 4: Characterization of Dispersion Stability of Pulse Protein Isolates

A protein dispersion stability study was carried out using isoelectric-precipitated isolate (IEP19) and ultra-filtered isolate (UF327) prepared according to the methods discussed in Example 1. The protein dispersion of 12% (w/w) protein isolate in water was homogenized with the PRO25D homogenizer (Pro Scientific, Oxford, Miss.) at 5000 rpm for 3 minutes, then 0.35% (w/w) salt (Culinox 999, Morton, Chicago, Ill.) was added and homogenized for another 2 minutes at 5000 rpm. 1 ml of the above dispersion was then pipetted into a 4 ml glass vessel (VIAL, 4 ml, clear glass, 15×45 mm, E&K Scientific Products, Santa Clara, Calif.) and closed with a screw cap. Two replicates were taken for each isolate of the protein-salt mixture. The glass vessels were refrigerated for 48 hrs at 4° C. before measuring the separation ratio. Separation ratio was defined as: Separation ratio=height of water phase/total height of sample.

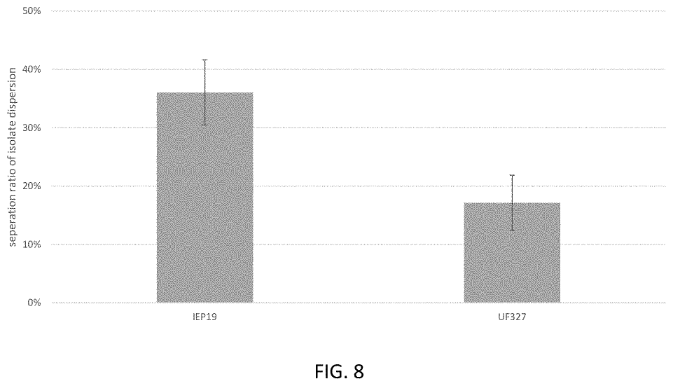

After the 48 hr storage at 4° C., the dispersions prepared with IEP isolate (IEP19) showed a clear separation with clear water layer on top and protein sedimentation in the bottom. In comparison, the dispersions prepared with the UF isolate (UF327) showed much less separation (n=8, p<0.01, Mann-Whitney U test). The separation ratio of the dispersions are shown in FIG. 8.

### Example 5: Characterization of Rheological Properties of Pulse Protein Isolates

The rheological properties of the IEP an UF isolates prepared according to the methods discussed in Example 1 were evaluated. Gelation of the protein isolates was characterized with dynamic oscillatory rheology. A rheometer (Discovery Hybrid Rheometer, TA instruments) equipped with a flat parallel plate geometry (40 mm diameter) was used to monitor each isolate's viscoelastic properties as a result of increasing temperature. Samples of isolate were prepared at 12% protein concentration. About 1.5 mL of sample was loaded onto the lower plate of the rheometer and was trimmed according to standard procedures. A solvent trap was loaded with 2 mL of distilled water to prevent evaporation of water within the sample as a result of the increase in temperature during the measurement. The storage (G′) and loss (G″) modulus were continuously recorded during a temperature ramp from 30° C. to 95° C. at a heating rate of 5° C./minute under small deformation conditions (0.1% strain) at a constant angular frequency of 10 rad/s followed by a 1 minute hold at 95° C. After this hold, the temperature of the material was reduced to 50° C. and an amplitude sweep test from 0.01 to 100% strain was carried out at a constant frequency of 10 rad/s in order to characterize the gelled material's linear viscoelastic region. Each sample was run in duplicate.

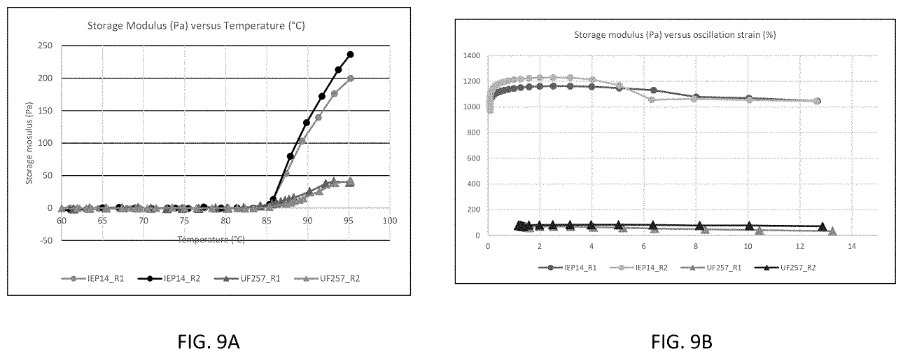

As shown in FIGS. 9A and 9B, the temperature ramp showed a similar on-set gelation temperature for the IEP and UF samples, but the IEP formed a stronger gel as storage modulus increased to a higher level (FIG. 9A), and the amplitude sweep showed a stronger solid-like behavior for the IEP sample at the linear visco-elastic region (FIG. 9B).

### Example 6: Characterization of Shelf-Stability of Food Product Composition Containing Pulse Protein Isolates

The following examples discusses a viscosity shelf-life study on an egg-free liquid egg analog product (JUST Egg) formulated by mixing either an isoelectric-precipitated (IEP) or ultra-filtered (UF) mung bean isolate with water, oil, emulsifiers and buffer salts. Various lots of the IEP or UF isolates were used in this study. After preparation, the formulated liquid egg analog products were immediately placed in a freezer at −18° C., thawed on the following day, and then stored under refrigeration at 4° C. Viscosity of the above-identified liquid egg analog products was measured at various time points throughout the study period (i.e., post freeze thaw day 0, day 30 and day 60 of refrigeration).

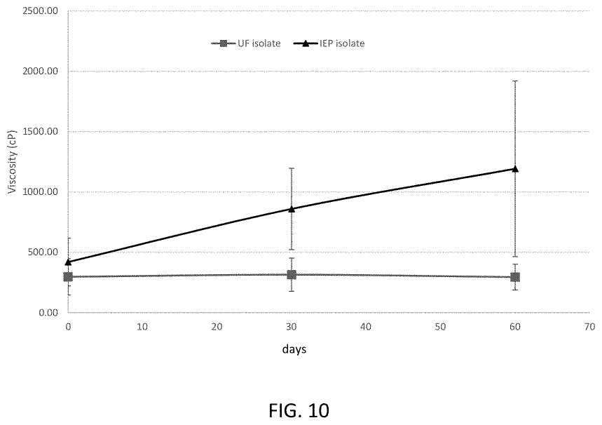

Viscosity was measured by a Brookfield DV-1 Prime viscometer with a flat disc RV spindle. Liquid egg analog samples were poured into a cylindrical 250 ml glass container immediately upon removal from the refrigerator. The reading was taken at 50 rpm and the temperature of samples was controlled below 8° C. while the reading was taken. The viscosity of liquid egg analog made from ultra-filtered isolates did not significantly change in comparison to day 0 and remained within the established product specifications, suggesting that the product is stable when stored for up to two months under refrigeration. Contrary to this finding, the viscosity of the liquid egg analog made from isoelectrically-precipitated isolates showed widely varying stability depending on the specific lot, with some showing increases in viscosity as high as 20 fold. In all lots tested, the IEP isolate-containing samples were significantly more viscous after storage for two months under refrigeration. FIG. 10 shows the average viscosity of the IEP and UF containing lots of liquid egg analog products tested.

The texture of the egg-free liquid egg analog was also shown to be superior when the ultrafiltered mung bean protein isolate used in the egg analog food product was prepared via a process including a first pH adjustment of the retentate fraction to a pH of 4.6, and a second pH adjustment to a pH of 6.0 (data not shown).

### Example 7: Ultrafiltration Process for Preparing Pulse Protein Isolates Using Different Molecular Weight Filters

The following example discusses an exemplary process for the production of an ultrafiltered (UF) pulse protein isolate.

Ultrafiltered Pulse Protein Isolate: 40 kg of Mung bean flour that was preprocessed by drying and grinding was extracted with 200 kg water, 600 g salt (NaCl), 100 mL antifoam in a Breddo liquefier (Corbion Inc). The mixing was performed for 2.5 minutes. The pH at the end of the run was adjusted to 7.0 using 1 M NaOH solution. The flour slurry was then centrifuged to perform a starch solid separation using a decanter (SG2-100, Alfalaval Inc). The major portion of the starch solids and unextracted material (decanter heavy phase) was separated from the liquid suspension (decanter light phase). The resuspension stream (light phase) was further clarified using a disc stack centrifuge (Clara 80, Alfalaval Inc.) into a high solids slurry (disc stack heavy phase) and a clarified resuspension (disc stack light phase). The disc stack heavy phase typically consists of fat, ash, starch and the protein carried over with the liquid portion of the slurry. The disc stack light phase (protein-rich fraction) was then processed through an ultrafiltration-diafiltration process with a custom designed membrane purification unit (Alfalaval Inc.). This unit was setup with three different membranes from Synder Inc. 10 kDa, 20 kDa, or 50 kDa.

The membrane material used is a polyether sulfone membrane purchased from Snyder, Inc. The disc stack light phase was initially processed in the membrane unit with the permeate returned to the feed tank to study the performance. There were slight differences in the rates between the different membranes when tested. Flux is the flow rate of the permeate through the membrane normalized to the total area of the membrane. TMP is the transmembrane pressure or the average pressure across the membrane module given that there is no pressure on the permeate line The rejection (ability to retain) of protein is quite different between the three membranes. As expected, the higher cut off membranes had a lower rejection coefficient. The process was continued at the highest TMP since it was still in the linear range and the light phase was concentrated from 75 kg to about 20 kg (3-4× concentration).

The concentrated protein suspension was further diafiltered with deionized water (DI) water in three steps adding about equal amounts of water at each step as the concentrate weight. The stream of diafiltered UF concentrate (19.5 kg) was then collected and the pH of this concentrate was adjusted from 7 to 6.1 using 20% w/w citric acid solution. Salt (NaCl) was added to adjust the conductivity in the 2-3 mS/cm range. The concentrate material was then heat treated using a microthermics UHT unit with the pasteurization condition set at 72.5° C. and 30 sec hold time. The heat-treated material was then spray dried to obtain protein isolate. The final protein recovery was about 95%, 80% and 75% from the 10 kDa, 20 kDa, and 50 kDa membranes, respectively.

### Example 8: Preparation and Texture Acceptability of Food Products Prepared from Mung Bean Protein Isolates

Plant-based egg substitutes, made with the protein isolates of Example 7, were prepared using a formula similar to a formula disclosed in Applicant's patent application WO2017/143298 (incorporated by reference). The plant-based egg substitutes were cooked into scrambled egg analogs and the texture acceptability of the plant-based egg substitutes were determined by a panel of 4 trained panelists. Texture acceptability is measured on a scale of 1-5 and the texture is acceptable if a score of 3.0 or above is reached. The plant-based scrambled egg analogs made with mung bean protein isolates prepared using a 10 kDa or 20 kDa membrane possessed acceptable texture but the egg analog made with the protein isolate prepared using the 50 kDa membrane was found to be unacceptable or marginally acceptable. Table 1 below discloses the results.

TABLE 1

| | | | Texture Acceptability |
|---|---|---|---|
| Membrane | Protein % | NaCl | (median ± median absolute deviation) |
| 10 kDa | 13 | 0.4% | 3.5 ± 0.5 |
| 20 kDa | 13 | 0.4% | 3.0 ± 0.5 |
| 50 kDa | 13 | 0.4% | 2.5 ± 0.5 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method for preparing a pulse protein isolate, comprising:
obtaining a milled composition;
extracting protein from the milled composition comprising pulse proteins in an aqueous solution at a pH of from about 1 to about 9 to produce a protein rich fraction containing extracted pulse proteins;
applying the protein rich fraction to an ultrafiltration process comprising a semi-permeable membrane to separate a retentate fraction from a permeate fraction based on molecular size at a temperature of from 2° C. to 60° C.; and
collecting the retentate fraction containing the pulse protein isolate.

2. The method of claim 1, further comprising adjusting the pH of the retentate fraction to a first pH of from 4.0 to 7.0, optionally followed by a further pH adjustment to a second pH of from 5.0 to 6.6.

3. The method of claim 2, wherein
(a) the first pH or the second pH of the retentate fraction is adjusted to a pH of from 5.8 to 6.6, or
(b) the first pH or the second pH of the retentate fraction is adjusted to a pH of from 6.0 to 6.2.

4. (canceled)

5. The method of claim 1, further comprising:
(a) heating the retentate fraction to a temperature of from 60° C. to 80° C. for a period of time from 10 seconds to 10 minutes, or
(b) heating the retentate fraction to a temperature of from 65° C. to 80° C. for a period of time from 10 seconds to 10 minutes, or
(c) heating the retentate fraction to a temperature of from 70° C. to 80° C. for a period of time from 10 seconds to 10 minutes, or
(d) heating the retentate fraction to a temperature of from 70° C. to 75° C. for a period of time from 10 seconds to 10 minutes, or
(e) heating the retentate fraction to a temperature of from 60° C. to 80° C. for a period of time from 10 seconds to 5 minutes, or
(f) heating the retentate fraction to a temperature of from 60° C. to 80° C. for a period of time from 10 seconds to 1 minute, or
(g) heating the retentate fraction to a temperature of from 60° C. to 80° C. for a period of time from 10 seconds to 30 seconds.

6-11. (canceled)

12. The method of claim 1, further comprising:
(a) removing water from the retentate fraction to produce a concentrated pulse protein isolate or
(b) removing water from the retentate fraction by spray drying, drum drying, tray drying, flash drying, or freeze drying.

13. (canceled)

14. The method of claim 1, wherein the milled composition comprising pulse proteins is a milled composition of:
(a) dry beans, lentils, faba beans, dry peas, chickpeas, cowpeas, bambara beans, pigeon peas, lupins, vetches, adzuki, common beans, fenugreek, long beans, lima beans, runner beans, tepary beans, or
(b) mung beans.

15. (canceled)

16. The method of claim 1, further comprising:
(a) dehulling pulses, milling pulses, or dehulling and milling pulses to produce the milled composition comprising pulse proteins, or
(b) dry-milling or wet-milling pulses, and/or
(c) drying the pulses prior to milling.

17-18. (canceled)

19. The method of claim 1:
(a) further comprising air classifying the milled composition prior to extracting protein, and/or
(b) further comprising applying the protein rich fraction to a pre-filtration process before applying the protein rich fraction to the ultrafiltration process, and/or
(c) wherein the pulse proteins are not precipitated from the protein rich fraction at a pH of from 4 to 6.

20-21. (canceled)

22. The method of claim 1, wherein:
(a) the retentate fraction comprises pulse proteins enriched in proteins having a molecular size of greater than 5 kilodaltons (kDa), 10 kDa, 20 kDa, 50 kDa or 75 kDa, but less than 100 kDa,
(b) the permeate fraction comprises pulse proteins depleted in proteins having a molecular size of less than 5 kilodaltons (kDa), 10 kDa, 20 kDa, 50 kDa or 75 kDa
(c) the retentate fraction comprises pulse proteins having a molecular size of less than 100 kilodaltons (kDa)
(d) the retentate fraction comprises pulse proteins having a molecular size of less than 50 kDa
(e) the retentate fraction comprises pulse proteins having a molecular size of less than 25 kDa, or
(f) the retentate fraction comprises pulse proteins having a molecular size of less than 15 kDa.

23-27. (canceled)

28. The method of claim 1, wherein the semi-permeable membrane:
(a) excludes molecules having a size of 1 kDa or larger,
(b) excludes molecules having a size of 3 kDa or larger,
(c) excludes molecules having a size of 5 kDa or larger,
(d) excludes molecules having a size of 7.5 kDa or larger,
(e) excludes molecules having a size of 10 kDa or larger,
(f) excludes molecules having a size of 20 kDa or larger,
(g) excludes molecules having a size of 30 kDa or larger,
(h) excludes molecules having a size of 50 kDa or larger,
(i) excludes molecules having a size of 70, 80, 90 or 95 kDa or larger,
(j) has a pore size of from 0.001 to 0.1 micron,
(k) has a pore size of from 0.001 to 0.006 micron,
(l) has a pore size of from 0.001 to 0.005 micron,
(m) has a pore size of from 0.0025 to 0.005 micron,
(n) has a pore size of about 0.003 micron,
(o) is a polymeric membrane, a ceramic membrane, or a metallic membrane, or
(p) is made from polyvinylidine fluoride (PVDF), polyether sulfone (PES), polyacrylonitrile (PAN), polytetrafluoroethylene (PTFE), polyamide-imide (PAI), a natural polymer, rubber, wool, cellulose, stainless steel, tungsten, palladium, an oxide, a nitride, a metallic carbide, aluminum carbide, titanium carbide, or a hydrated aluminosilicate mineral containing an alkali and alkaline-earth metal.

29-43. (canceled)

44. The method of claim 1, wherein:
(a) the ultrafiltration process is performed at a pressure of from about 20 to about 500 psig,
(b) the aqueous solution comprises a salt,
(c) the aqueous solution comprises a salt at a concentration of from 0.01% w/v to 5% w/v,
(d) the aqueous solution comprises a salt at a concentration of from 0.001% w/v to 0.1% w/v, 0.001% w/v to 0.2% w/v, 0.001% w/v to 0.3% w/v, or 0.001% w/v to 0.4% w/v,
(e) the aqueous solution comprises a salt at a concentration of from 0.1% w/v to 0.5% w/v, 0.1% w/v to 1% w/v, 1.0% w/v to 2.5% w/v, or 2.5% w/v to 5% w/v,
(f) the aqueous solution comprises a salt selected from sodium chloride, sodium sulfate, sodium phosphate, ammonium sulfate, ammonium phosphate, ammonium chloride, potassium chloride, potassium sulfate, or potassium phosphate,
(g) the aqueous solution comprises a NaCl, or
(h) the aqueous solution does not comprise a salt.

45-51. (canceled)

52. The method of claim 1, wherein the density of the pulse protein isolate:
(a) is less than 0.6 g/ml,
(b) is less than 0.5 g/ml or 0.4 g/ml,
(c) is less than 0.3 g/ml, or
(d) is less than 0.2 g/ml or 0.1 g/ml.

53-55. (canceled)

56. The method of claim 1, wherein a homogenized protein dispersion consisting of 12% w/w pulse protein isolate, 0.35% w/w NaCl, and water has a separation ratio of:
(a) less than 30% after 48 hours of storage at 4° C.,
(b) less than 25% after 48 hours of storage at 4° C., or
(c) less than 20% after 48 hours of storage at 4° C.

57-58. (canceled)

59. The method of claim 1, wherein the pulse protein isolate:
(a) has a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s,
(b) has a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C., and/or
(c) has a linear viscoelastic region of less than 500 Pa at up to 10% strain, or a linear viscoelastic region of less than 200 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C.

60-61. (canceled)

62. A pulse protein isolate prepared by the method of claim 1, or a food composition comprising a pulse protein isolated prepared by the method of claim 1 and one or more edible ingredients.

63. (canceled)

64. An isolated pulse protein having a density of less than 0.6 g/ml.

65. The isolated pulse protein of claim 64, wherein:
(a) the density is less than 0.5 g/ml or 0.4 g/ml,
(b) the density is less than 0.3 g/ml,
(c) the density is less than 0.2 g/ml or 0.1 g/ml,
(d) the pulse protein has a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s,
(e) the pulse protein has a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C., and/or
(f) the pulse protein has a linear viscoelastic region of less than 500 Pa at up to 10% strain, or a linear viscoelastic region of less than 200 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C.

66-70. (canceled)

71. The isolated pulse protein of claim 64, wherein pulse protein is isolated from:
(a) dry beans, lentils, faba beans, dry peas, chickpeas, cowpeas, bambara beans, pigeon peas, lupins, vetches, adzuki, common beans, fenugreek, long beans, lima beans, runner beans, or tepary beans, or
(b) mung beans.

72. (canceled)

73. The isolated pulse protein of claim 64, wherein the pulse protein:
(a) is enriched in proteins having a molecular size of greater than 5 kilodaltons (kDa), 10 kDa, 20 kDa, 50 kDa or 75 kDa, but less than 100 kDa,
(b) is depleted in proteins having a molecular size of less than 5 kilodaltons (kDa), 10 kDa, 20 kDa, 50 kDa or 75 kDa,
(c) includes proteins having a molecular size of less than 100 kDa,
(d) includes proteins having a molecular size of less than 50 kDa,
(e) includes proteins having a molecular size of less than 25 kDa,
(f) includes proteins having a molecular size of less than 15 kDa, or
(g) includes proteins having a molecular size of from 1 kDa to 99 kDa.

74-79. (canceled)

80. A food composition comprising a pulse protein isolate of claim 64, and one or more edible ingredients, wherein:
(a) the composition has a viscosity of less than 500 cP after storage for thirty days at 4° C.,
(b) the composition has a viscosity of less than 500 cP after storage for sixty days at 4° C.,
(c) the composition has a viscosity of less than 450 cP after storage for thirty days at 4° C.,
(d) the composition has a viscosity of less than 450 cP after storage for sixty days at 4° C., or
(e) the composition comprises pulse proteins that are depleted in proteins having a molecular size of less than 10 kilodaltons (kDa) or 20 kDa, and wherein the food composition has improved texture as compared to a reference food composition comprising proteins depleted in proteins having a molecular size of less than 50 kilodaltons (kDa), 75 kDa, or 100 kDa.

81-85. (canceled)
